SEQUENCE LISTING (12 sequences)

SEQ ID NO: 1 - DNA, 673 bp, Nicotiana tabacum
gcggccgctaaacctccttgttcttttacgccagaggaagctgaatatttaacatctcgtatacaaaatgggggaactgaagttgttgaggcaaaagctggtgctggttcggcaactctctctatggcatatgctgcggttaaatttgccgacgcatgtttgcatggattgagaggagatgctggcattgtagaatgtgcctttgtgtcttctcaggtgactgaacttccatttttcgcatcaaaagtatggcttggccgcaacggagttgaagaaatatacccccttggtcccctaaatgaatacgagaggtctgggcttgagaaggcaaggaaagagttggcaacaagtgttcagaagggtgtcaactttgtaaagaaatgagcagacagctacatgacttccaaaagatgcttttatgtgggctatatatctcaaatccgcagttccagaaaataagagtagtttctttcttgtattaaagggcaaatcctgttctaattttctatagattgatgccttggtgcagaaaataaatgtactatttggtcatctaaaataacaacagtccccagtgcatgttggacttgcaaagtattacatcctttgaagcaagggcttgttatggactttttgacagtatggatatttaaagggcttggagagcggccgc

SEQ ID NO: 2 - DNA, 1505 bp, Nicotiana tabacum
  CDS (148)..(1221)
ctaatacgactcactatagggcaagcagtggtatcaacgcagagtacgcgggggggaaacaaaattcaattacttaccttgatttctactacctctctttctcatcataattcaaacacacaaattctcaagcccaagtcttagaatatgcagaacggtgcagagacctatcgacgaatggccaccatctcagctcaccttaacccctctccttcttctcatcagatggagggaggtgtgggtttgagccgagctaattgcagggcgaaagggggttctccaggattcaaagtcgcgatcttgggtgctgcaggaggtattggtcagccacttgctatgcttatgaaaacgaatccactggtttcagttctgcatctttatgatgttgccaatactcctggtgtaactgctgacattagccacatggacactggtgccgtggtacgtggttttctagggcctcaacaattggaagatgctctcactggcatggaccttgtaataatccctgctggtgttcctagaaaaccaggcatgacaagagatgatcttttcaacatcaatgcaggaattgtgaggactttatgtgaaggaattgccaagtgctgtcctaaggccattgttaacataattagtaatcctgttaactctacagtaccaattgctgcagaggttttcaagaaggctggcacctttgatccgaggagactgttgggcgtgacaatgcttgatattgtcagagccaatacatttgtggctgaagttttggggcttgatcctagggaagtggatgttccagttgtggggggtcatgctggcgttacaattctacctcttctttcccaggttaaacctccttgttcttttacgccagaggaaactgaatatttaacatctcgtatacaaaatgggggaactgaagttgttgaggcaaaagctggtgctggttcggcaactctctctatggcatatgctgcggttaaatttgccgacgcatgtttgcatggattgagaggagatgctggcattgtagaatgtgcctttgtgtcttctcaggtgactgaacttccatttttcgcatcaaaagtacggcttggccgcaacggagttgaagaaatatacccccttggtcccctaaatgaatacgagaggtctgggcttgagaaggcaaagaaagagctggcaacaagtgttcagaagggtgtcaactttgtaaagaaatgagcagacagctacatgacttccaaaagatgcttttatgtgggctatatatctcaaatccgcagttccagaaaataagagtagtttctttcttgtattaaagggcaaatcctgttctaattttctatagattgatgccttggtgcagaaaataaatgtactatttggtcatctaaaataacaacagtccccagtgcatgttggacttgcaaagtattacatcctttgaagcaagggcttgttatggactttttgacagtatggatatttaaagggcttggaga

SEQ ID NO: 3 - protein, 358 aa, Nicotiana tabacum
MQNGAETYRRMATISAHLNPSPSSHQMEGGVGLSRANCRAKGGSPGFKVAILGAAGGIGQPLAMLMKTNPLVSVLHLYDVANTPGVTADISHMDTGAVVRGFLGPQQLEDALTGMDLVIIPAGVPRKPGMTRDDLFNINAGIVRTLCEGIAKCCPKAIVNIISNPVNSTVPIAAEVFKKAGTFDPRRLLGVTMLDIVRANTFVAEVLGLDPREVDVPVVGGHAGVTILPLLSQVKPPCSFTPEETEYLTSRIQNGGTEVVEAKAGAGSATLSMAYAAVKFADACLHGLRGDAGIVECAFVSSQVTELPFFASKVRLGRNGVEEIYPLGPLNEYERSGLEKAKKELATSVQKGVNFVKK

SEQ ID NO: 4 - DNA, 16 bp, Artificial Sequence
  Primer
agaattcgcggccgct

SEQ ID NO: 5 - DNA, 32 bp, Artificial Sequence
  Primer
ctcatgcggccgcgcgcaacgcaattaatgtg

SEQ ID NO: 6 - DNA, 32 bp, Artificial Sequence
  Primer
tcatgcggccgcgagatccagttcgatgtaac

SEQ ID NO: 7 - DNA, 21 bp, Artificial Sequence
  Primer
gtggattgatgtgatatctcc

SEQ ID NO: 8 - DNA, 21 bp, Artificial Sequence
  Primer
gtaaggatctgagctacacat

SEQ ID NO: 9 - DNA, 17 bp, Artificial Sequence
  Primer
atgagggcgaaaggggg

SEQ ID NO: 10 - DNA, 25 bp, Artificial Sequence
  Primer
tttctttacaaagttgacacccttc

SEQ ID NO: 11 - DNA, 17 bp, Artificial Sequence
  Primer
atgcgggcaaaaggtgg

SEQ ID NO: 12 - DNA, 21 bp, Artificial Sequence
  Primer
tttcttcgcaaaggtaacacc

(19) United States
(12) Patent Application Publication
(10) Pub. No.: US 2006/0058190 A1
(43) Pub. Date: Mar. 16, 2006

(54) Malate dehydrogenase as a target for herbicides

(76) Inventors: Thomas Ehrhardt, Speyer (DE); Andreas Reindl, Mannheim (DE); Annette Freund, Limburgerhof (DE); Ralf-Michael Schmidt, Kirrweiler (DE); Kirsten Deist, Westdorf (DE); Uwe Sonnewald, Quedlinburg (DE); Marc Stitt Nigel, Potsdam (DK); Wolfgang Lein, Potsdam (DE); Frederik Bornke, Quedlinburg (DE)

Correspondence Address: HUTCHISON & MASON PLLC, PO BOX 31686, RALEIGH, NC, 27612, US

(21) Appl. No.: 10/538,544

(22) PCT Filed: Dec. 17, 2003
(86) PCT No.: PCT/EP03/14379

§ 371 Date: Jun. 10, 2005

(30) Foreign Application Priority Data
Dec. 20, 2002 (DE) 10261192.0

(51) Int. Cl. A01N 25/00; C12Q 1/48

(52) U.S. Cl. 504/116.1; 435/15

(57) ABSTRACT

The present invention relates to malate dehydrogenase, which, when absent, brings about reduced growth and chlorotic leaves and is encoded by the nucleic acid sequence SEQ ID NO:2 or by a functional equivalent of this nucleic acid sequence, as target for herbicides. For this purpose, novel nucleic acid sequences encompassing SEQ ID NO:2 and functional equivalents of SEQ ID NO:2 are provided. Moreover, the present invention relates to the use of the abovementioned malate dehydrogenase and its functional equivalents in a method for identifying compounds with herbicidal or growth-regulatory activity, and to the use of the compounds identified by this method as herbicides or growth regulators.

The present invention relates to malate dehydrogenase, which, when absent, brings about reduced growth and chlorotic leaves and is encoded by the nucleic acid sequence SEQ ID NO:2 or by a functional equivalent of this nucleic acid sequence, as target for herbicides. For this purpose, novel nucleic acid sequences encompassing SEQ ID NO:2 and functional equivalents of SEQ ID NO:2 are provided. Moreover, the present invention relates to the use of the abovementioned malate dehydrogenase and its functional equivalents in a method for identifying compounds with herbicidal or growth-regulatory activity, and to the use of the compounds identified by this method as herbicides or growth regulators.

The basic principle of identifying herbicides via the inhibition of a defined target is known (for example U.S. Pat. No. 5,187,071, WO 98/33925, WO 00/77185). In general, there is a great demand for the detection of enzymes which might constitute novel targets for herbicides. The reasons are resistance problems which occur with herbicidal active ingredients which act on known targets, and the ongoing endeavor to identify novel herbicidal active ingredients which are distinguished by as wide as possible a spectrum of action, ecological and toxicological acceptability and/or low application rates.

In practice, the detection of novel targets entails great difficulties since the inhibition of an enzyme which forms part of a metabolic pathway frequently has no further effect on the growth of the plant. This may be attributed to the fact that the plant switches to alternative metabolic pathways whose existence is not known or that the inhibited enzyme is not limiting for the metabolic pathway. Furthermore, plant genomes are distinguished by a high degree of functional redundancy. Functionally equivalent enzymes are found more frequently in gene families in the Arabidopsis thaliana genome than in insects or mammals (Nature, 2000, 408(6814):796-815). This hypothesis is confirmed experimentally by the fact that comprehensive gene knock-out programs by T-DNA or transposon insertion into Arabidopsis yielded fewer manifested phenotypes to date than expected (Curr. Op. Plant Biol. 4, 2001, pp. 111-117).

It is an object of the present invention to identify novel targets which are essential for the growth of plants or whose inhibition leads to reduced plant growth, and to provide methods which are suitable for identifying herbicidally active and/or growth-regulatory-compounds.

We have found that this object is achieved by the use of malate dehydrogenase in a method for identifying herbicides.

Further terms used in the description are now defined at this point.

“Affinity tag”: this refers to a peptide or polypeptide whose coding nucleic acid sequence can be fused to the nucleic acid sequence according to the invention either directly or by means of a linker, using customary cloning techniques. The affinity tag serves for the isolation, concentration and/or selective purification of the recombinant target protein by means of affinity chromatography from total cell extracts. The abovementioned linker can advantageously contain a protease cleavage site (for example for thrombin or factor Xa), whereby the affinity tag can be cleaved from the target protein when required. Examples of common affinity tags are the “His tag”, for example from Qiagen, Hilden, “Strep tag”, the “Myc tag” (Invitrogen, Carlsberg), the tag from New England Biolabs which consists of a chitin-binding domain and an inteine, the maltose-binding protein (pMal) from New England Biolabs, and what is known as the CBD tag from Novagen. In this context, the affinity tag can be attached to the 5′ or the 3′ end of the coding nucleic acid sequence with the sequence encoding the target protein.

“Biological activity of a glyoxysomal malate dehydrogenase”: for the purposes of the present invention, this term describes the presence of the glyoxysomal malate dehydrogenase being able to confer the ability to grow and viability. If the activity of a protein with the biological activity of a glyoxysomal malate dehydrogenase is inhibited, this leads to reduced growth, growth stop or death of the plant.

“Enzymatic activity of a glyoxysomal malate dehydrogenase”: the term enzymatic activity describes the ability of an enzyme to convert a substrate into a product. The enzymatic activity can be determined in what is known as an activity assay via the increase in the product, the decrease in the substrate (or starting material) or the decrease in a specific cofactor, or via a combination of at least two of the abovementioned parameters, as a function of a defined period of time.

“Enzymatic activity of a glyoxysomal malate dehydrogenase” describes here the ability of an enzyme to catalyze a reaction which is likewise catalyzed by the glyoxysomal malate dehydrogenase.

“Expression cassette”: an expression cassette contains a nucleic acid sequence according to the invention linked operably to at least one genetic control element, such as a promoter, and, advantageously, a further control element, such as a terminator. The nucleic acid sequence of the expression cassette can be for example a genomic or complementary DNA sequence or an RNA sequence, and their semisynthetic or fully synthetic analogs. These sequences can exist in linear or circular form, extrachromosomally or integrated into the genome. The nucleic acid sequences in question can be synthesized or obtained naturally or contain a mixture of synthetic and natural DNA components, or else consist of various heterologous gene segments of various organisms.

Artificial nucleic acid sequences are also suitable in this context as long as they make possible the expression, in a cell or an organism, of a polypeptide with the enzymatic activity of a glyoxysomal malate dehydrogenase, preferably with the biological activity of a glyoxysomal malate dehydrogenase, which polypeptide is encoded by a nucleic acid sequence according to the invention. For example, synthetic nucleotide sequences can be generated-which have been optimized with regard to the codon-usage of the organisms to be transformed.

All of the abovementioned nucleotide sequences can be generated from the nucleotide units by chemical synthesis in the manner known per se, for example by fragment condensation of individual overlapping complementary nucleotide units of the double helix. Oligonucleotides can be synthesized chemically for example in the manner known per se using the phosphoamidite method (Voet, Voet, 2^{nd} Edition, Wiley Press New York, pp. 896-897). When preparing an expression cassette, various DNA fragments can be manipulated in such a way that a nucleotide sequence with the correct direction of reading and the correct reading frame is obtained. The nucleic acid fragments are linked with each other via general cloning techniques as are described, for example, in T. Maniatis, E. F. Fritsch and J. Sambrook, “Molecular Cloning: A Laboratory Manual”, Cold Spring Harbor Laboratory, Cold Spring Harbor, N.Y. (1989), in T. J. Silhavy, M. L. Berman and L. W. Enquist, Experiments with Gene Fusions, Cold Spring Harbor Laboratory, Cold Spring Harbor, N.Y. (1984) and in Ausubel, F.M. et al., “Current Protocols in Molecular Biology”, Greene Publishing Assoc. and Wiley-Interscience (1994).

“Operable linkage”: an operable, or functional, linkage is understood as meaning the sequential arrangement of regulatory sequences or genetic control elements in such a way that each of the regulatory sequences, or each of the genetic control elements, can fulfill its intended function when the coding sequence is expressed.

“Functional equivalents” describe, in the present context, nucleic acid sequences which hybridize under standard conditions with the nucleic acid sequence SEQ ID NO:2 or parts of SEQ ID NO:2 and which are capable of bringing about the expression, in a cell or an organism, of a polypeptide with the enzymatic activity of a glyoxysomal malate dehydrogenase, preferably with biological activity of a malate dehydrogenase.

To carry out the hybridization, it is advantageous to use short oligonucleotides with a length of approximately 10-50 bp, preferably 15-40 bp, for example of the conserved or other regions, which can be determined in the manner with which the skilled worker is familiar by comparisons with other related genes. However, longer fragments of the nucleic acids according to the invention with a length of 100-500 bp, or the complete sequences, may also be used for hybridization. Depending on the nucleic acid/oligonucleotide used, the length of the fragment or the complete sequence, or depending on which type of nucleic acid, i.e. DNA or RNA, is being used for the hybridization, these standard conditions vary. Thus, for example, the melting temperatures for DNA:DNA hybrids are approximately 10° C. lower than those of DNA:RNA hybrids of the same length.

Standard hybridization conditions are to be understood as meaning, depending on the nucleic acid, for example temperatures of between 42 and 58° C. in an aqueous buffer solution with a concentration of between 0.1 and 5×SSC (1×SSC=0.15 M NaCl, 15 mM sodium citrate, pH 7.2) or additionally in the presence of 50% formamide, such as, for example, 42° C. in 5×SSC, 50% formamide. The hybridization conditions for DNA:DNA hybrids are advantageously 0.1×SSC and temperatures of between approximately 20° C. and 65° C., preferably between approximately 30° C. and 45° C. In the case of DNA:RNA hybrids, the hybridization conditions are advantageously 0.1×SSC and temperatures of between approximately 30° C. and 65° C., preferably between approximately 45° C. and 55° C. These hybridization temperatures which have been stated are melting temperature values which have been calculated by way of example for a nucleic acid with a length of approx. 100 nucleotides and a G+C content of 50% in the absence of formamide. The experimental conditions for DNA hybridization are described in relevant textbooks of genetics such as, for example, in Sambrook et al., “Molecular Cloning”, Cold Spring Harbor Laboratory, 1989, and can be calculated using formulae with which the skilled worker is familiar, for example as a function of the length of nucleic acids, the type of the hybrids or the G+C content. The skilled worker will find further information on hybridization in the following textbooks: Ausubel et al. (eds), 1985, “Current Protocols in Molecular Biology”, John Wiley & Sons, New York; Hames and Higgins (eds.), 1985, “Nucleic Acids Hybridization: A Practical Approach”, IRL Press at Oxford University Press, Oxford; Brown (ed.), 1991, Essential Molecular Biology: A Practical Approach, IRL Press at Oxford University Press, Oxford.

A functional equivalent of SEQ ID NO:2 is furthermore also understood as meaning nucleic acid sequences having a defined degree of homology or identity with SEQ ID NO:2, and furthermore in particular also natural or artificial mutations of the abovementioned nucleic acid sequences which encode a polypeptide with the enzymatic, preferably the biological, activity of a glyoxysomal malate dehydrogenase.

The present invention also encompasses, for example, those nucleotide sequences which are obtained by modification of the abovementioned nucleic acid sequences. For example, such modifications can be generated by techniques with which the skilled worker is familiar, such as “Site Directed Mutagenesis”, “Error Prone PCR”, “DNA-shuffling” (Nature 370, 1994, pp. 389-391) or “Staggered Extension Process” (Nature Biotechnol. 16, 1998, pp. 258-261). The aim of such a modification can be, for example, the insertion of further cleavage sites for restriction enzymes, the removal of DNA in order to truncate the sequence, the substitution of nucleotides to optimize the codons, or the addition of further sequences. Proteins which are encoded via modified nucleic acid sequences must retain the desired function despite a deviating nucleic acid sequence.

The term “functional equivalents” can also relate to the amino acid sequence encoded by the nucleic acid sequence in question. In this case, the term “functional equivalent” describes a protein whose amino acid sequence has a defined percentage of identity or homology with SEQ ID NO:3.

Functional equivalents thus comprise naturally occurring variants of the herein-described sequences and artificial nucleic acid sequences, for example those which have been obtained by chemical synthesis and which are adapted to the codon usage, and also the amino acid sequences derived from them.

“Genetic control sequence” describes sequences which have an effect on the transcription and, if appropriate, translation of the nucleic acids according to the invention in prokaryotic or eukaryotic organisms. Examples thereof are promoters, terminators or what are known as “enhancer” sequences. In addition to these control sequences, or instead of these sequences, the natural regulation of these sequences may still be present before the actual structural genes and may, if appropriate, have been genetically modified in such a way that the natural regulation has been switched off and the expression of the target gene has been modified, that is to say increased or reduced. The choice of the control sequence depends on the host organism or starting organism. Genetic control sequences furthermore also comprise the 5′-untranslated region, introns or the noncoding 3′-region of genes. Control sequences are furthermore understood as meaning those which make possible homologous recombination or insertion into the genome of a host organism or which permit removal from the genome. Genetic control sequences also comprise further promoters, promoter elements or minimal promoters, and sequences which have an effect on the chromatin structure (for example matrix attachment regions (MARs)), which can modify the expression-governing properties. Thus, genetic control sequences may bring about for example the additional dependence of the tissue-specific expression on certain stress factors. Such elements have been described, for example, for water stress, abscisic acid (Lam E and Chua N H, J Biol Chem 1991; 266(26): 17131-17135), high- and low-temperature stress (Plant Cell 1994, (6): 251-264) and heat stress (Molecular & General Genetics, 1989, 217 (2-3): 246-53).

“Homology” between two nucleic acid sequences or polypeptide sequences is defined by the identity of the nucleic acid sequence/polypeptide sequence over in each case the entire sequence length, which is calculated by alignment with the aid of the program algorithm BESTFIT (Wisconsin Package Version 10.0, University of Wisconsin, Genetics Computer Group (GCG), Madison, USA), setting the following parameters for polypeptides:

| | |
|---|---|
| Gap Weight: 8 | Length Weight: 2 |
| Average Match: 2,912 | Average Mismatch: −2,003 |

and the following parameters for nucleic acids:

| | |
|---|---|
| Gap Weight: 50 | Length Weight: 3 |
| Average Match: 10.000 | Average Mismatch: −9.000 |

In the following text, the term identity is also used synonymously with the term “homologous” or “homology”.

“Mutations” of nucleic or amino acid sequences comprise substitutions, additions, deletions, inversions or insertions of one or more nucleotide residues, which may also bring about changes in the corresponding amino acid sequence of the target protein by substitution, insertion or deletion of one or more amino acids, although the functional properties of the target proteins are, overall, essentially retained.

“Natural genetic environment” means the natural chromosomal locus in the organism of origin. In the case of a genomic library, the natural genetic environment of the nucleic acid sequence is preferably retained at least in part. The environment flanks the nucleic acid sequence at least at the 5′- or 3′-side and has a sequence length of at least 50 bp, preferably at least 100 bp, especially preferably at least 500 bp, very especially preferably at least 1000 bp, and most preferably at least 5000 bp.

“Plants” for the purposes of the invention are plant cells, plant tissues, plant organs, or intact plants, such as seeds, tubers, flowers, pollen, fruits, seedlings, roots, leaves, stems or other plant parts. Moreover, the term plants is understood as meaning propagation material such as seeds, fruits, seedlings, slips, tubers, cuttings or root stocks.

“Reaction time” refers to the time required for carrying out an assay for determining the enzymatic activity until a significant finding regarding an enzymatic activity is obtained; it depends both on the specific activity of the protein employed in the assay and on the method used and the sensitivity of the instruments used. The skilled worker is familiar with the determination of the reaction times. In the case of methods for identifying herbicidally active compounds which are based on photometry, the reaction times are, for example, generally between >0 to 120 minutes.

“Recombinant DNA” describes a combination of DNA sequences which can be generated by recombinant DNA technology.

“Recombinant DNA technology”: generally known techniques for fusing DNA sequences (for example described in Sambrook et al., 1989, Cold Spring Harbor, N.Y., Cold Spring Harbor Laboratory Press).

“Replication origins” ensure the multiplication of the expression cassettes or vectors according to the invention in microorganisms and yeasts, for example the pBR322 ori or the P15A ori in E. coli (Sambrook et al.: “Molecular Cloning. A Laboratory Manual”, 2nd ed. Cold Spring Harbor Laboratory Press, Cold Spring Harbor, N.Y., 1989) and the ARS1 ori in yeast (Nucleic Acids Research, 2000, 28(10): 2060-2068).

“Reporter genes” encode readily quantifiable proteins. The transformation efficacy or the expression site or timing can be assessed by means of these genes via growth assay, fluorescence assay, chemoluminescence assay, bioluminescence assay or resistance assay or via a photometric measurement (intrinsic color) or enzyme activity. Very especially preferred in this context are reporter proteins (Schenborn E, Groskreutz D. Mol Biotechnol. 1999; 13(1):29-44) such as the “green fluorescence protein” (GFP) (Gerdes H H and Kaether C, FEBS Lett. 1996; 389(1):44-47; Chui W L et al., Curr Biol 1996, 6:325-330; Leffel S M et al., Biotechniques. 23(5):912-8, 1997), chloramphenicol acetyl transferase, a luciferase (Giacomin, Plant Sci 1996, 116:59-72; Scikantha, J Bact 1996, 178:121; Millar et al., Plant Mol Biol Rep 1992 10:324-414), and luciferase genes, in general β-galactosidase or β-glucuronidase (Jefferson et al., EMBO J. 1987, 6, 3901-3907) or the Ura3 gene.

“Selection markers” confer resistance to antibiotics or other toxic compounds: examples which may be mentioned in this context are the neomycin phosphotransferase gene, which confers resistance to the aminoglycoside antibiotics neomycin (G 418), kanamycin, paromycin (Deshayes A et al., EMBO J. 4 (1985) 2731-2737), the sul gene, which encodes a mutated dihydropteroate synthase (Guerineau F et al., Plant Mol Biol. 1990; 15(1):127-136), the hygromycin B phosphotransferase gene (Gen Bank Accession NO: K 01193) and the shble resistance gene, which confers resistance to the bleomycin antibiotics such as zeocin. Further examples of selection marker genes are genes which confer resistance to 2-deoxyglucose-6-phosphate (WO 98/45456) or phosphinothricin and the like, or those which confer a resistance to antimetabolites, for example the dhfr gene (Reiss, Plant Physiol. (Life Sci. Adv.) 13 (1994) 142-149). Examples of other genes which are suitable are trpB or hisD (Hartman S C and Mulligan R C, Proc Natl Acad Sci USA. 85 (1988) 8047-8051). Another suitable gene is the mannose phosphate isomerase gene (WO 94/20627), the ODC (ornithine decarboxylase) gene (McConlogue, 1987 in: Current Communications in Molecular Biology, Cold Spring Harbor Laboratory, Ed.) or the Aspergillus terreus deaminase (Tamura K et al., Biosci Biotechnol Biochem. 59 (1995) 2336-2338).

“Transformation” describes a process for introducing heterologous DNA into a pro- or eukaryotic cell. The term transformed cell describes not only the product of the transformation process per se, but also all of the transgenic progeny of the transgenic organism generated by the transformation.

“Target/target protein”: a polypeptide encoded via the nucleic acid sequence according to the invention, which may take the form of an enzyme in the traditional sense or, for example, of a structural protein, a protein relevant for developmental processes, regulatory protein such as transcription factors, kinases, phosphatases, receptors, channel subunits, transport proteins, regulatory subunits which confer substrate or activity regulation to an enzyme complex. All of the targets or sites of action share the characteristic that their functional presence is essential for survival or normal development and growth.

“Transgenic”: referring to a nucleic acid sequence, an expression cassette or a vector comprising a nucleic acid sequence according to the invention or an organism transformed with the abovementioned nucleic acid sequence, expression cassette or vector, the term transgenic describes all those constructs which have been generated by genetic engineering methods in which either the nucleic acid sequence of the target protein or a genetic control sequence linked operably to the nucleic acid sequence of the target protein or a combination of the abovementioned possibilities are not in their natural genetic environment or have been modified by recombinant methods. In this context, the modification can be achieved, for example, by mutating one or more nucleotide residues of the nucleic acid sequence in question.

Malate dehydrogenase catalyzes the reversible, pyridine nucleotide-dependent conversion of oxalacetate and malate. Owing to the conversion of these important metabolites of the primary metabolism, the reaction catalyzed by malate dehydrogenase impacts on a variety of processes (Review in Faske et al. 1997, Plant Physiology 115, 705-715). NAD⁺-dependent malate dehydrogenases can be found in mitochondria, microbodies such as peroxysomes or glyoxysomes and in the cytosol and in root knots, while chloroplasts contain an NADP⁺-dependent malate dehydrogenase whose regulation is light-dependent. In Arabidopsis, malate dehydrogenases are encoded by a gene family. The individual isoforms contain specific N-terminal transit sequences for transport into the chloroplasts, mitochondria and microbodies. There are furthermore sequences for malate dehydrogenases which, owing to the absence of transit peptides, presumably remain in the cytosol. Sequences encoding malate dehydrogenases have been isolated from a variety of C3 and C4 plants (see Miller et al. 1998 Plant Journal 15, 173-184). Nucleic acid sequences encoding glyoxysomal malate dehydrogenase, and protein sequences, are known to exist in watermelon (P19446, Swissprot; identity with SEQ ID NO:2=77.5%; identity with Seq ID NO:3=85.4%), cucumber, Arabidopsis, soybean and rice.

The antisense inhibition of a cytosolic malate dehydrogenase in potato down to 40% residual activity resulted in no pronounced growth phenotypes and no reduced tuber yield (Jenner et al. 2001, Plant Physiology 126, 1139-1149). According to Faske et al. (1997, Plant Physiology 115, 705-715), reduced expression of a tobacco plastid malate dehydrogenase by antisense and cosuppression has no effect on the vitality of the transgenic plants, even when malate dehydrogenase expression is greatly reduced (<20% residual activity). Only minor changes with regard to the growth parameters (fresh weight, dry weight) were observed. Attempts to overexpress a Sorghum plastid malate dehydrogenase in the C4 plant Flaveria by cosuppression resulted in a drastic inhibition of the malate dehydrogenase activity down to 5-50% residual activity (Trevanion et al. 1997, Plant Physiology 113, 1153-1165) without having significant effects on plant vitality.

Surprisingly, it has been found within the scope of the present invention that plants in which glyoxysomal malate dehydrogenase was reduced in a selective manner have phenotypes which are comparable with phenotypes generated by herbicide application. Drastic growth retardation and damage such as chloroses and necroses were observed.

Although cytosolic and plastid malate dehydrogenase is not essential for plants, these enzymes can, in principle, also be used to determine inhibitors of the glyoxysomal malate dehydrogenase, since the reaction catalyzed by the cytosolic and plastid malate dehydrogenase is the same as the reaction catalyzed by the glyoxysomal malate dehydrogenase.

The present invention relates to the use of malate dehydrogenase in a method for identifying herbicides, preferably of plant malate dehydrogenase, especially preferably plant malate dehydrogenase encoded by a nucleic acid sequence comprising

a) a nucleic acid sequence with the nucleic acid sequence shown in SEQ ID NO:1, or

b) a nucleic acid sequence with the nucleic acid sequence shown in SEQ ID NO:2, or

c) a nucleic acid sequence which, owing to the degeneracy of the genetic code, can be deduced from the amino acid sequence shown in SEQ ID NO:3 by backtranslating, or

d) a nucleic acid sequence which, owing to the degeneracy of the genetic code, can be deduced by backtranslating the amino acid sequence of a functional equivalent of SEQ ID NO:3, which has at least 63% identity with SEQ ID NO:3,

where the functional equivalents of c) and d) are distinguished by the same functionality, i.e. they have the enzymatic activity of glyoxsysomal malate dehydrogenase.

The term “comprising” or “to comprise”, referring to nucleic acid sequences, means that the nucleic acid sequence according to the invention can comprise additional nucleic acid sequences at the 3′ or the 5′ end, where the length of the additional nucleic acid sequences does not exceed 500 bp at the 5′ end and 500 bp at the 3′ end of the nucleic acid sequences according to the invention, preferably 250 bp at the 5′ end and 250 bp at the 3′ end, especially preferably 100 bp at the 5′ end and 100 bp at the 3′ end.

Examples of functional equivalents according to d) are also the plant nucleic acid sequences encoding malate dehydrogenase from
cucumber (Gen Bank Accession Number: L31900)
Arabidopsis (Gen Bank Accession Number: AC005671)
soybean (Gen Bank Accession Number: L01628) and
rice (Gen Bank Accession Number: D85763)
   and the nucleic acid sequence which is encoded by the amino acid sequence from a malate dehydrogenase from watermelon (Accession Number: P19446, Swissprot).

All of the abovementioned sequences are likewise subject matter of the present invention.

The functional equivalent according to the invention of SEQ ID NO:3 as described in d) has at least 63%, 64%, 65%, 66%, by preference at least 67%, 68%, 69%, 70%, 71%, 72%, 73%, 74%, with preference at least 75%, 76%, 77%, 78%, 79%, 80%, preferably at least 81%, 82%, 83%, 84%, 85%, 86%, 87%, 88%, 89%, 89%, 90%, 91%, 92%, 93%, very especially preferably at least 94%, 95%, 96%, 97%, 98%, 99%, homology with SEQ ID NO:3.

Especially preferred is the use of glyoxysomal malate dehydrogenase in a method for identifying herbicides encoded by a nucleic acid sequence comprising

a) a nucleic acid sequence with the nucleic acid sequence shown in SEQ ID NO:1, or

b) a nucleic acid sequence with the nucleic acid sequence shown in SEQ ID NO:2, or

c) a nucleic acid sequence which, owing to the degeneracy of the genetic code, can be deduced from the amino acid sequence shown in SEQ ID NO:3 by backtranslating, or

d) a nucleic acid sequence which, owing to the degeneracy of the genetic code, can be deduced by backtranslating the amino acid sequence of a functional equivalent of SEQ ID NO:3, which has at least 66% identity with SEQ ID NO:3.

The functional equivalents as described in d) are distinguished by the same functionality, i.e. they have the biological function of a glyoxysomal malate dehydrogenase.

The abovementioned nucleic acid sequences originate by preference from a plant.

The functional equivalent according to the invention of SEQ ID NO:3 as described in d) has at least 66%, 67%, 68%, by preference at least 69%, 70%, 71%, 72%, 73%, 74%, 75%, 76%, 77%, 78%, 79%, 80%, with preference at least 81%, 82%, 83%, preferably at least 84%, 85%, 86%, 87%, 88%, 89%, 89%, 90%, 91%, 92%, 93%, very especially preferably at least 94%, 95%, 96%, 97%, 98%, 99%, homology with SEQ ID No.3.

Furthermore claimed within the scope of the present invention are nucleic acid sequences encoding a plant glyoxysomal malate dehydrogenase comprising:

a) a nucleic acid sequence with the nucleic acid sequence shown in SEQ ID NO:1, or

b) a nucleic acid sequence with the nucleic acid sequence shown in SEQ ID NO:2, or

c) a nucleic acid sequence which, owing to the degeneracy of the genetic code, can be deduced from the amino acid sequence shown in SEQ ID NO:3 by backtranslating, or

d) functional equivalents of the nucleic acid sequence SEQ ID NO:2 with at least 79% identity with SEQ ID NO:2; or

e) a nucleic acid sequence which, owing to the degeneracy of the genetic code, can be deduced by backtranslating the amino acid sequence of a functional equivalent of SEQ ID NO:3, which has at least 87% identity with SEQ ID NO:3.

The polypeptides encoded by the abovementioned nucleic acid sequences are likewise claimed. The functional equivalents as described in c) and d) are distinguished by the same functionality, i.e. they have the enzymatic, preferably biological, activity of a glyoxysomal malate dehydrogenase.

The functional equivalents of SEQ ID NO:2 have. at least 79%, 80%, 81%, 82%, 83%, by preference at least 84%, 85%, 86%, 88%, 89%, preferably at least 90%, 91%, 92%, 93%, 94%, 95%, especially preferably at least 96%, 97%, 98%, 99%, identity with SEQ ID NO:3.

The functional equivalents according to the invention of SEQ ID NO:3 have at least 87%, 88%, 89%, by preference at least 90%, 91%, 92%, 93%, preferably at least 94%, 95%, 96%, especially preferably at least 97%, 98%, 99%, identity with SEQ ID NO:3.

The term “nucleic acid sequences according to the invention” which is used hereinbelow refers to nucleic acid sequences encoding a malate dehydrogenase, by preference nucleic acid sequences encoding a plant malate dehydrogenase, especially preferably nucleic acid sequences encoding a plant malate dehydrogenase comprising

a) a nucleic acid sequence with the nucleic acid sequence shown in SEQ ID NO:1, or

b) a nucleic acid sequence with the nucleic acid sequence shown in SEQ ID NO:2, or

c) a nucleic acid sequence which, owing to the degeneracy of the genetic code, can be deduced from the amino acid sequence shown in SEQ ID NO:3 by backtranslating, or

d) a nucleic acid sequence which, owing to the degeneracy of the genetic code, can be deduced by backtranslating the amino acid sequence of a functional equivalent of SEQ ID NO:3, which has at least 63% identity with SEQ ID NO:3,

very especially preferably nucleic acid sequences encoding a plant glyoxysomal malate dehydrogenase comprising

a) a nucleic acid sequence with the nucleic acid sequence shown in SEQ ID NO:1, or

b) a nucleic acid sequence with the nucleic acid sequence shown in SEQ ID NO:2, or

c) a nucleic acid sequence which, owing to the degeneracy of the genetic code, can be deduced from the amino acid sequence shown in SEQ ID NO:3 by backtranslating, or

d) a nucleic acid sequence which, owing to the degeneracy of the genetic code, can be deduced by backtranslating the amino acid sequence of a functional equivalent of SEQ ID NO:3, which has at least 66% identity with SEQ ID NO:3.

The polypeptides encoded by a nucleic acid sequence according to the invention which have the enzymatic, preferably biological, activity of a glyoxsysomal malate dehydrogenase are hereinbelow simply referred to as “MDH”. The term “malate dehydrogenase” refers to any protein/enzyme with the enzymatic activity of a malate dehydrogenase.

Reduced amounts of glyoxysomal MDHs cause growth retardation and necrotic and chlorotic leaves in plants.

The gene products of the nucleic acids according to the invention constitute novel targets for herbicides, which make possible the provision of novel herbicides for controlling undesired plants. Moreover, the gene products of the nucleic acids according to the invention constitute novel targets for growth regulators which make possible the provision of novel growth regulators for regulating the growth of plants.

Undesired plants are understood as meaning, in the broadest sense, all those plants which grow at locations where they are undesired, for example:

Dicotyledonous weeds of the genera: Sinapis, Lepidium, Galium, Stellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, Taraxacum.

Monocotyledonous weeds from the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristylis, Sagittaria, Eleocharis, Scirpus, Paspalum, Ischaemum, Sphenoclea, Dactyloctenium, Agrostis, Alopecurus, Apera.

SEQ ID NO:2 or parts of SEQ ID NO:2 can be used for the preparation of hybridization probes. The preparation of these probes and the experimental procedure is known. For example, this can be effected via the selective preparation of radioactive or nonradioactive probes by PCR and the use of suitably labeled oligonucleotides, followed by hybridization experiments. The technologies required for this purpose are detailed, for example, in T. Maniatis, E. F. Fritsch and J. Sambrook, “Molecular Cloning: A Laboratory Manual”, Cold Spring Harbor Laboratory, Cold Spring Harbor, N.Y. (1989). The probes in question can furthermore be modified by standard technologies (Lit. SDM or random mutagenesis) in such a way that they can be employed for further purposes, for example as a probe which hybridizes specifically with mRNA and the corresponding coding sequences in order to analyze the corresponding sequences in other organisms.

The abovementioned probes can be used for the detection and isolation of functional equivalents of SEQ ID NO:2 from other plant species on the basis of sequence identities. In this context, part or all of the sequence of the SEQ ID NO:2 in question is used as a probe for screening a genomic or cDNA library of the plant species in question or in a computer search for sequences of functional equivalents in electronic databases.

Preferred plant species are the undesired plants which have already been mentioned at the outset.

The invention furthermore relates to expression cassettes comprising

a) genetic control sequences in operable linkage with a nucleic acid sequence according to the invention comprising
i. a nucleic acid with the nucleic acid sequence shown in SEQ ID NO:2, or
ii. a nucleic acid sequence which, owing to the degeneracy of the genetic code, can be deduced from the amino acid sequence shown in SEQ ID NO:3 by backtranslation, or
iii. functional equivalents of the nucleic acid sequence SEQ ID NO:2 with at least 79% identity to SEQ ID NO:2,
iv. a nucleic acid sequence which, owing to the degeneracy of the genetic code, can be deduced by backtranslating the amino acid sequence of a functional equivalent of SEQ ID NO:3, which has at least 92% identity with SEQ ID NO:3,

b) additional functional elements, or

c) a combination of a) and b);

and to the use of expression cassettes comprising

a) genetic control sequences in operable linkage with a nucleic acid sequence according to the invention,

b) additional functional elements, or

c) a combination of a) and b);

for expressing a malate dehydrogenase, preferably MDH, which can be used in in vitro assay systems. Both embodiments of the above-described expression cassettes are referred in the following text as expression cassette according to the invention.

In a preferred embodiment, an expression cassette according to the invention comprises a promoter at the 5′ end of the coding sequence and, at the 3′ end, a transcription termination signal and, if appropriate, further genetic control sequences which are linked operably with the interposed nucleic acid sequence according to the invention.

The expression cassettes according to the invention are also understood as meaning analogs which can be brought about, for example, by a combination of the individual nucleic acid sequences on a polynucleotide (multiple constructs), on a plurality of polynucleotides in a cell (cotransformation) or by sequential transformation.

Advantageous genetic control sequences under point a) for the expression cassettes according to the invention or for vectors comprising expression cassettes according to the invention are, for example, promoters such as the cos, tac, trp, tet, lpp, lac, lacIq, T7, T5, T3, gal, trc, ara, SP6, λ-PR or the λ-PL promoter, all of which can be used for expressing a malate dehydrogenase, preferably MDH, in Gram-negative bacterial strains.

Examples of further advantageous genetic control sequences are present, for example, in the promoters amy and SPO2, both of which can be used for expressing a malate dehydrogenase, preferably MDH, in Gram-positive bacterial strains, and in the yeast or fungal promoters AUG1, GPD-1, PX6, TEF, CUP1, PGK, GAP1, TPI, PHO5, AOX1, GAL10/CYC1, CYC1, OliC, ADH, TDH, Kex2, MFA or NMT or combinations of the abovementioned promoters (Degryse et al., Yeast June 1995 15; 11(7):629-40; Romanos et al. Yeast June 1992; 8(6):423-88; Benito et al. Eur. J. Plant Pathol. 104, 207-220 (1998); Cregg et al. Biotechnology (N Y) August 1993; 11(8):905-10; Luo X., Gene September 1995 22;163(1):127-31: Nacken et al., Gene October 1996 10;175(1-2): 253-60; Turgeon et al., Mol Cell Biol September 1987; 7(9):3297-305) or the transcription terminators NMT, Gcy1, TrpC, AOX1, nos, PGK or CYC1 (Degryse et al., Yeast June 1995 15; 11(7):629-40; Brunelli et al. Yeast 1993 (Dec9(12): 1309-18; Frisch et al., Plant Mol. Biol. 27(2), 405-409 (1995); Scorer et al., Biotechnology (N.Y. 12 (2), 181-184 (1994), Genbank acc. number Z46232; Zhao et al. Genbank acc number: AF049064; Punt et al., (1987) Gene 56 (1), 117-124), all of which can be used for expressing malate dehydrogenase, preferably MDH, in yeast strains.

Examples of genetic control sequences which are suitable for expression in insect cells are the polyhedrin promoter and the p10 promoter (Luckow, V. A. and Summers, M. D. (1988) Bio/Techn. 6, 47-55).

Advantageous genetic control sequences for expressing malate dehydrogenase, preferably MDH, in cell culture, in addition to polyadenylation sequences such as, for example, from simian virus 40, are eukaryotic promoters of viral origin such as, for example, promoters of the polyoma virus, adenovirus 2, cytomegalovirus or simian virus 40.

Further advantageous genetic control sequences for expressing malate dehydrogenasee, preferably MDH, in plants are present in the plant promoters CaMV/35S [Franck et al., Cell 21(1980) 285-294], PRP1 [Ward et al., Plant. Mol. Biol. 22 (1993)], SSU, OCS, LEB4, USP, STLS1, B33, NOS; FBPaseP (WO 98/18940) or in the ubiquitin or phaseolin promoter; a promoter which is preferably used being, in particular, a plant promoter or a promoter derived from a plant virus. Especially preferred are promoters of viral origin such as the promoter of the cauliflower mosaic virus 35S transcript (Franck et al., Cell 21 (1980), 285-294; Odell et al., Nature 313 (1985), 810-812). Further preferred constitutive promoters are, for example, the agrobacterium nopaline synthase promoter, the TR double promoter, the agrobacterium OCS (octopine synthase) promoter, the ubiquitin promoter, (Holtorf S et al., Plant Mol Biol 1995, 29:637-649), the promoters of the vacuolar ATPase subunits, or the promoter of a proline-rich wheat protein (WO 91/13991).

The expression cassettes may also comprise, as genetic control sequence, a chemically inducible promoter, by which the expression of the exogenous gene in the plant can be controlled at a specific point in time. Such promoters, such as, for example, the PRP1 promoter (Ward et al., Plant. Mol. Biol. 22 (1993), 361-366), a salicylic-acid-inducible promoter (WO 95/19443), a benzenesulfonamide-inducible promoter (EP-A-0388186), a tetracyclin-inducible promoter (Gatz et al., (1992) Plant J. 2, 397404), an abscisic-acid-inducible promoter (EP-A 335528) or an ethanol- or cyclohexanone-inducible promoter (WO 93/21334) may also be used.

Furthermore, suitable promoters are those which confer tissue- or organ-specific expression in, for example, anthers, ovaries, flowers and floral organs, leaves, stomata, trichomes, stems, vascular tissues, roots and seeds. Others which are suitable in addition to the abovementioned constitutive promoters are, in particular, those promoters which ensure leaf-specific expression. Promoters which must be mentioned are the potato cytosolic FBPase promoter (WO 97/05900), the rubisco (ribulose-1,5-bisphosphate carboxylase) SSU (small subunit) promoter or the ST-LSI promoter from potato (Stockhaus et al., EMBO J. 8 (1989), 2445-245). Promoters which are furthermore preferred are those which control expression in seeds and plant embryos. Examples of seed-specific promoters are the phaseolin promoter (U.S. Pat. No. 5,504,200, Bustos M M et al., Plant Cell. 1989;1(9):839-53), the promoter of the 2S albumin gene (Joseffson L G et al., J Biol Chem 1987, 262:12196-12201), the legumin promoter (Shirsat A et al., Mol Gen Genet. 1989;215(2):326-331), the USP (unknown seed protein) promoter (Bäumlein H et al., Molecular & General Genetics 1991, 225(3):459-67), the napin gene promoter (Stalberg K, et al., L. Planta 1996, 199:515-519), the sucrose binding protein promoter (WO 00/26388) or the LeB4 promoter (Bäumlein H et al., Mol Gen Genet 1991, 225: 121-128; Fiedler, U. et al., Biotechnology (NY) (1995), 13 (10) 1090).

Further promoters which are suitable as genetic control sequences are, for example, specific promoters for tubers, storage roots or roots, such as, for example, the class I patatin promoter (B33), the potato cathepsin D inhibitor promoter, the starch synthase (GBSS1) promoter or the sporamin promoter, fruit-specific promoters such as, for example, the fruit-specific promoter from tomato (EP-A 409625), fruit-maturation-specific promoters such as, for example, the fruit-maturation-specific promoter from tomato (WO 94/21794), inflorescence-specific promoters such as, for example, the phytoene synthase promoter (WO 92/16635) or the promoter of the P-rr gene (WO 98/22593), or plastid- or chromoplast-specific promoters such as, for example, the RNA polymerase promoter (WO 97/06250), or else the Glycine max phosphoribosyl-pyrophosphate amidotransferase promoter (see also Genbank Accession No. U87999), or another node-specific promoter as described in EP-A 249676.

Additional functional elements b) are understood as meaning, by way of example but not by limitation, reporter genes, replication origins, selection markers and what are known as affinity tags, in fusion with malate dehydrogenase, preferably MDH, directly or by means of a linker optionally comprising a protease cleavage site. Further suitable additional functional elements are sequences which ensure that the product is targeted into the apoplasts, into plastids, the vacuole, the mitochondrion, the peroxisome, the endoplasmatic reticulum (ER) or, owing to the absence of such operative sequences, remains in the compartment where it is formed, the cytosol, (Kermode, Crit. Rev. Plant Sci. 15, 4 (1996), 285-423).

Also in accordance with the invention are vectors comprising at least one copy of the nucleic acid sequences according to the invention and/or the expression cassettes according to the invention.

In addition to plasmids, vectors are furthermore also understood as meaning all of the other known vectors with which the skilled worker is familiar, such as, for example, phages, viruses such as SV40, CMV, baculovirus, adenovirus, transposons, IS elements, phasmids, phagemids, cosmids or linear or circular DNA. These vectors can be replicated autonomously in the host organism or replicated chromosomally; chromosomal replication is preferred.

In a further embodiment of the vector, the nucleic acid construct according to the invention can advantageously also be introduced into the organisms in the form of a linear DNA and integrated into the genome of the host organism via heterologous or homologous recombination. This linear DNA may consist of a linearized plasmid or only of the nucleic acid construct as vector, or the nucleic acid sequences used.

Further prokaryotic or eukaryotic expression systems are mentioned in Chapters 16 and 17 in Sambrook et al., “Molecular Cloning: A Laboratory Manual.” 2nd ed., Cold Spring Harbor Laboratory, Cold Spring Harbor Laboratory Press, Cold Spring Harbor, N.Y., 1989. Further advantageous vectors are described in Hellens et al. (Trends in plant science, 5, 2000).

The expression cassette according to the invention and vectors derived therefrom can be used for transforming bacteria, cyanobacteria, (for example of the genus Synechocystis, Anabaena, Calothrix, Scytonema, Oscillatoria, Plectonema and Nostoc), proteobacteria such as, for example, Magnetococcus sp. MC1, yeasts, filamentous fungi and algae and eukaryotic nonhuman cells (for example insect cells) with the aim of producing malate dehydrogenase, preferably MDH, recombinantly, the generation of a suitable expression cassette depending on the organism in which the gene is to be expressed.

Vectors Comprising

a) a nucleic acid sequence with the nucleic acid sequences as shown in SEQ ID NO:2, or

b) a nucleic acid sequence which, owing to the degeneracy of the genetic code, can be produced from the amino acid sequence as shown in SEQ ID NO:3 by backtranslation, or

c) functional equivalents of the nucleic acid sequence SEQ ID NO:2 with at least 79% identity with SEQ ID NO:2;

d) a nucleic acid sequence which, owing to the degeneracy of the genetic code, can be deduced by backtranslating the amino acid sequence of a functional equivalent of SEQ ID NO:3 which has at least 87% identity with SEQ ID NO:3,

form part of the subject-matter of the present invention.

In a further advantageous embodiment, the nucleic acid sequences used in the method according to the invention may also be introduced into an organism by themselves.

If, in addition to the nucleic acid sequences, further genes are to be introduced into the organism, they can all be introduced into the organism together in a single vector, or each individual gene can be introduced into the organism in each case in one vector, it being possible to introduce the different vectors simultaneously or in succession.

In this context, the introduction, into the organisms in question (transformation), of the nucleic acid(s) according to the invention, of the expression cassette or of the vector can be effected in principle by all methods with which the skilled worker is familiar.

In the case of microorganisms, the skilled worker will find suitable methods in the textbooks by Sambrook, J. et al. (1989) “Molecular cloning: A laboratory manual”, Cold Spring Harbor Laboratory Press, von F. M. Ausubel et al. (1994) “Current protocols in molecular biology”, John Wiley and Sons, by D. M. Glover et al., DNA Cloning Vol.1, (1995), IRL Press (ISBN 019-963476-9), by Kaiser et al. (1994) Methods in Yeast Genetics, Cold Spring Harbor Laboratory Press or Guthrie et al. “Guide to Yeast Genetics and Molecular Biology”, Methods in Enzymology, 1994, Academic Press. In the transformation of filamentous fungi, the methods of choice are firstly the generation of protoplasts and transformation with the aid of PEG (Wiebe et al. (1997) Mycol. Res. 101 (7): 971-877; Proctor et al. (1997) Microbiol. 143, 2538-2591), and secondly the transformation with the aid of Agrobacterium tumefaciens (de Groot et al. (1998) Nat. Biotech. 16, 839-842).

In the case of dicots, the methods which have been described for the transformation and regeneration of plants from plant tissues or plant cells can be exploited for transient or stable transformation. Suitable methods are the biolistic method or by transformation of protoplasts (cf., for example, Willmitzer, L., 1993 Transgenic plants. In: Biotechnology, A Multi-Volume Comprehensive Treatise (H. J. Rehm, G. Reed, A. Pühler, P. Stadler, eds.), Vol. 2, 627-659, VCH Weinheim-New York-Basle-Cambridge), electroporation, the incubation of dry embryos in DNA-containing solution, microinjection and the agrobacterium-radiated gene transfer. The abovementioned methods are described, for example, in B. Jenes et al., Techniques for Gene Transfer, in: Transgenic Plants, Vol. 1, Engineering and Utilization, edited by S. D. Kung and R. Wu, Academic Press (1993) 128-143 and in Potrykus, Annu. Rev. Plant Physiol. Plant Molec. Biol. 42 (1991) 205-225).

The transformation by means of agrobacteria, and the vectors to be used for the transformation, are known to the skilled worker and described extensively in the literature (Bevan et al., Nucl. Acids Res. 12 (1984) 8711. The intermediary vectors can be integrated into the agrobacterial Ti or Ri plasmid by means of homologous recombination owing to sequences which are homologous to sequences in the T-DNA. This plasmid additionally contains the vir region, which is required for the transfer of the T-DNA. Intermediary vectors are not capable of replication in agrobacteria. The intermediary vector can be transferred to Agrobacterium tumefaciens by means of a helper plasmid (conjugation). Binary vectors are capable of replication both in E. coli and in agrobacteria. They contain a selection marker gene and a linker or polylinker which are framed by the right and left T-DNA border region. They can be transformed directly into the agrobacteria (Holsters et al. Mol. Gen. Genet. 163 (1978), 181-187), EP A 0 120 516; Hoekema, in: The Binary Plant Vector System Offsetdrukkerij Kanters B. V., Alblasserdam (1985), Chapter V; Fraley et al., Crit. Rev. Plant. Sci., 4: 1-46 and An et al. EMBO J. 4 (1985), 277-287).

The transformation of monocots by means of vectors based on agrobacterium has also been described (Chan et al., Plant Mol. Biol. 22(1993), 491-506; Hiei et al., Plant J. 6 (1994) 271-282; Deng et al. Science in China 33 (1990), 28-34; Wilmink et al., Plant Cell Reports 11, (1992) 76-80; May et al. Biotechnology 13 (1995) 486-492; Conner and Domisse; Int. J. Plant Sci. 153 (1992) 550-555; Ritchie et al. Transgenic Res. (1993) 252-265). Alternative systems for the transformation of monocots are the transformation by means of biolistic approach (Wan and Lemaux; Plant Physiol. 104 (1994), 37-48; Vasil et al. Biotechnology 11 (1992), 667-674; Ritala et al., Plant Mol. Biol 24, (1994) 317-325; Spencer et al., Theor. Appl. Genet. 79 (1990), 625-631), protoplast transformation, the electroporation of partially permeabilized cells, and the introduction of DNA by means of glass fibers. In particular the transformation of maize has been described repeatedly in the literature (cf., for example, WO 95/06128; EP 0513849 A1; EP 0465875 A1; EP 0292435 A1; Fromm et al., Biotechnology 8 (1990), 833-844; Gordon-Kamm et al., Plant Cell 2 (1990), 603-618; Koziel et al., Biotechnology 11(1993) 194-200; Moroc et al., Theor Applied Genetics 80 (190) 721-726).

The successful transformation of other cereal species has also already been described for example in the case of barley (Wan and Lemaux, see above; Ritala et al., see above; wheat (Nehra et al., Plant J. 5(1994) 285-297).

Agrobacteria which have been transformed with a vector according to the invention can likewise be used in a known manner for the transformation of plants, such as test plants like Arabidopsis or crop plants like cereals, maize, oats, rye, barley, wheat, soybean, rice, cotton, sugarbeet, canola, sunflower, flax, hemp, potato, tobacco, tomato, carrot, capsicum, oilseed rape, tapioca, cassava, arrowroot, Tagetes, alfalfa, lettuce and the various tree, nut and grapevine species, for example by bathing scarified leaves or leaf segments in an agrobacterial solution and subsequently growing them in suitable media.

The genetically modified plant cells can be regenerated via all methods with which the skilled worker is familiar. Such methods can be found in the abovementioned publications by S. D. Kung and R. Wu, Potrykus or Höfgen and Willmitzer.

The transgenic organisms generated by transformation with one of the above-described embodiments of an expression cassette comprising a nucleic acid sequence according to the invention or a vector comprising the abovementioned expression cassette, and the recombinant malate dehydrogenase, preferably MDH, which can be obtained from the transgenic organism by means of expression, form part of the subject matter of the present invention. The use of transgenic organisms comprising an expression cassette according to the invention, for example for providing recombinant protein, and/or the use of these organisms in in-vivo assay systems likewise form part of the subject matter of the present invention.

Preferred organisms for the recombinant expression are not only bacteria, yeasts, mosses, algae and fungi, but also eukaryotic cell lines.

Preferred mosses are Physcomitrella patens or other mosses described in Kryptogamen [Cryptogamia], Vol.2, Moose, Farne [Mosses, Ferns], 1991, Springer Verlag (ISBN 3540536515).

Preferred within the bacteria are, for example, bacteria from the genus Escherichia, Erwinia, Flavobacterium, Alcaligenes or cyanobacteria, for example from the genus Synechocystis, Anabaena, Calothrix, Scytonema, Oscillatoria, Plectonema and Nostoc, especially preferably Synechocystis or Anabaena.

Preferred yeasts are Candida, Saccharomyces, Schizosaccheromyces, Hansenula or Pichia.

Preferred fungi are Aspergillus, Trichoderma, Ashbya, Neurospora, Fusarium, Beauveria, Mortierella, Saprolegnia, Pythium, or other fungi described in Indian Chem Engr. Section B. Vol 37, No 1,2 (1995).

Preferred plants are selected in particular among monocotyledonous crop plants such as, for example, cereal species such as wheat, barley, sorghum or millet, rye, triticale, maize, rice or oats, and sugarcane. The transgenic plants according to the invention are, furthermore, in particular selected from among dicotyledonous crop plants such as, for example, Brassicaceae such as oilseed rape, cress, Arabidopsis, cabbages or canola; Leguminosae such as soybean, alfalfa, pea, beans or peanut, Solanaceae such as potato, tobacco, tomato, egg plant or capsicum; Asteraceae such as sunflower, Tagetes, lettuce or Calendula; Cucurbitaceae such as melon, pumpkin/squash or zucchini, or linseed, cotton, hemp, flax, red pepper, carrot, sugar beet, or various tree, nut and grapevine species.

In principle, transgenic animals such as, for example, C. elegans, are also suitable as host organisms.

Also preferred is the use of expression systems and vectors which are available to the public or commercially available.

Those which must be mentioned for use in E. coli bacteria are the typical advantageous commercially available fusion and expression vectors pGEX [Pharmacia Biotech Inc; Smith, D. B. and Johnson, K. S. (1988) Gene 67:31-40], pMAL (New England Biolabs, Beverly, Mass.) and pRIT5 (Pharmacia, Piscataway, N.J.), which contains glutathione S transferase (GST), maltose binding protein or protein A, the pTrc vectors (Amann et al., (1988) Gene 69:301-315), “pKK233-2” from CLONTECH, Palo Alto, Calif. and the “pET”, and the “pBAD” vector series from Stratagene, La Jolla.

Further advantageous vectors for use in yeast are pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), and pYES derivatives, PGAPZ derivatives, pPICZ derivatives, and the vectors of the “Pichia Expression Kit” (Invitrogen Corporation, San Diego, Calif.). Vectors for use in filamentous fungi are described in: van den Hondel, C.A.M.J.J. & Punt, P. J. (1991) “Gene transfer systems and vector development for filamentous fungi, in: Applied Molecular Genetics of Fungi, J. F. Peberdy, et al., eds., p. 1-28, Cambridge University Press: Cambridge.

As an alternative, insect cell expression vectors may also be used advantageously, for example for expression in Sf9, Sf21 or Hi5 cells, which are infected via recombinant Baculoviruses. Examples of these are the vectors of the pAc series (Smith et al. (1983) Mol. Cell Biol. 3:2156-2165) and the pVL series (Lucklow and Summers (1989) Virology 170:31-39). Others which may be mentioned are the Baculovirus expression systems “MaxBac 2.0 Kit” and “Insect Select System” from Invitrogen, Carlsbad or “BacPAK Baculovirus Expression System” from CLONTECH, Palo Alto, Calif. Insect cells are particularly suitable for overexpressing eukaryotic proteins since they effect posttranslational modifications of the proteins which are not possible in bacteria and yeasts. The skilled worker is familiar with the handling of cultured insect cells and with their infection for expressing proteins, which can be carried out analogously to known methods (Luckow and Summers, Bio/Tech. 6, 1988, pp.47-55; Glover and Hames (eds) in DNA Cloning 2, A practical Approach, Expression Systems, Second Edition, Oxford University Press, 1995, 205-244).

Plant cells or algal cells are others which can be used advantageously for expressing genes. Examples of plant expression vectors can be found as mentioned above in Becker, D., et al. (1992) “New plant binary vectors with selectable markers located proximal to the left border”, Plant Mol. Biol. 20: 1195-1197 or in Bevan, M. W. (1984) “Binary Agrobacterium vectors for plant transformation”, Nucl. Acid. Res. 12: 8711-8721.

Moreover, the nucleic acid sequences according to the invention can be expressed in mammalian cells. Examples of suitable expression vectors are pCDM8 and pMT2PC, which are mentioned in: Seed, B. (1987) Nature 329:840 or Kaufman et al. (1987) EMBO J. 6:187-195). Promoters preferably to be used in this context are of viral origin such as, for example, promoters of polyoma virus, adenovirus 2, cytomegalovirus or simian virus 40. Further prokaryotic and eukaryotic expression systems are mentioned in Chapter 16 and 17 in Sambrook et al., Molecular Cloning: A Laboratory Manual. 2nd ed., Cold Spring Harbor Laboratory, Cold Spring Harbor Laboratory Press, Cold Spring Harbor, N.Y., 1989. Further advantageous vectors are described in Hellens et al. (Trends in plant science, 5, 2000).

The transgenic organisms which comprise a nucleic acid sequence comprising are claimed within the scope of the present invention.

All of the above-described embodiments of the transgenic organisms come under the term “transgenic organism according to the invention”.

The present invention furthermore relates to the use of malate dehydrogenase, preferably MDH, in a method for identifying herbicidally active test compounds.

The method according to the invention for identifying herbicidally active compounds preferably comprises the following steps:

i. bringing malate dehydrogenase, preferably MDH, into contact with one or more test compounds under conditions which permit binding of the test compound(s) to malate dehydrogenase, preferably MDH, and

ii. detecting whether the test compound binds to malate dehydrogenase, preferably the MDH of i); or

iii. detecting whether the test compound reduces or blocks the enzymatic or biological activity of malate dehydrogenase, preferably the MDH of i); or

iv. detecting whether the test compound reduces or blocks the transcription, translation or expression of a malate dehydrogenase, preferably MDH.

The detection in accordance with step (ii) of the above method can be effected using techniques which identify the interaction between protein and ligand. In this context, either the test compound or the enzyme can contain a detectable label such as, for example, a fluorescent label, a radioisotope, a chemiluminescent label or an enzyme label. Examples of enzyme labels are horseradish peroxidase, alkaline phosphatase or luciferase. The subsequent detection depends on the label and is known to the skilled worker.

In this context, five preferred embodiments which are also suitable for high-throughput methods (HTS) in connection with the present invention must be mentioned in particular:

1. The average diffusion rate of a fluorescent molecule as a function of the mass can be determined in a small sample volume via fluorescence correlation spectroscopy (FCS) (Proc. Natl. Acad. Sci. USA (1994) 11753-11575). FCS can be employed for determining protein/ligand interactions by measuring the change in the mass, or the changed diffusion rate which this entails, of a test compound when binding to malate dehydrogenase, preferably MDH. A method according to the invention can be designed directly for measuring the binding of a test compound labeled by a fluorescent molecule. As an alternative, the method according to the invention can be designed in such a way that a chemical reference compound which is labeled by a fluorescent molecule is displaced by further test compounds (“displacement assay”).

2. Fluoresence polarization exploits the characteristic of a quiescent fluorophore excited with polarized light to likewise emit polarized light. If, however, the fluorophore is allowed to rotate during the excited state, the polarization of the fluorescent light which is emitted is more or less lost. Under otherwise identical conditions (for example temperature, viscosity, solvent), the rotation is a function of molecule size, whereby findings regarding the size of the fluorophore-bound residue can be obtained via the reading (Methods in Enzymology 246 (1995), pp. 283-300). A method according to the invention can be designed directly for measuring the binding of a test compound labeled with a fluorescent molecule to the malate dehydrogenase, preferably MDH. As an alternative, the method according to the invention may also take the form of the “displacement assay” described under 1.

3. Fluorescence resonance energy transfer (FRET) is based on the irradiation-free energy transfer between two spatially adjacent fluorescent molecules under suitable conditions. A prerequisite is that the emission spectrum of the donor molecule overlaps with the excitation spectrum of the acceptor molecule. The fluorescent label of malate dehydrogenase, preferably MDH, and binding test compound, the binding can be measured by means of FRET (Cytometry 34, 1998, pp. 159-179). As an alternative, the method according to the invention may also take the form of the “displacement assay” described under 1. An especially suitable embodiment of FRET technology is “Homogeneous Time Resolved Fluorescence” (HTRF) as can be obtained from Packard BioScience.

4. Surface-enhanced laser desorption/ionization (SELDI) in combination with a time-of-flight mass spectrometer (MALDI-TOF) makes possible the rapid analysis of molecules on a support and can be used for analyzing protein/ligand interactions (Worral et al., (1998) Anal. Biochem. 70:750-756). In a preferred embodiment, malate dehydrogenase, preferably MDH, is immobilized on a suitable support and incubated with the test compound. After one or more suitable wash steps, the test compound molecules which are additionally bound to malate dehydrogenase, preferably MDH, can be detected by means of the abovementioned methodology and test compounds which are bound to malate dehydrogenase, preferably MDH, can thus be selected.

5. The measurement of surface plasmon resonance is based on the change in the refractive index at a surface when a test compound binds to a protein which is immobilized to said surface. Since the change in the refractive index is identical for virtually all proteins and polypeptides for a defined change in the mass concentration at the surface, this method can be applied to any protein in principle (Lindberg et al. Sensor Actuators 4 (1983) 299-304; Malmquist Nature 361 (1993) 186-187). The measurement can be carried out for example with the automatic analyzer based on surface plasmon resonance which is available from Biacore (Freiburg) at a throughput of, currently, up to 384 samples per day. A method according to the invention can be designed directly for measuring the binding of a test compound to malate dehydrogenase, preferably MDH. As an alternative, the method according to the invention may also take the form of the “displacement assay” described under 1.

The compounds identified via the abovementioned methods 1 to 5 may be suitable as inhibitors. All of the substances identified via the abovementioned methods can subsequently be checked for their herbicidal action in another embodiment of the method according to the invention.

Furthermore, there exists the possibility of detecting further candidates for herbicidal active ingredients by molecular modeling via elucidation of the three-dimensional structure of malate dehydrogenase, preferably MDH, by x-ray structure analysis. The preparation of protein crystals required for x-ray structure analysis, and the relevant measurements and subsequent evaluations of these measurements, the detection of a binding site in the protein, and the prediction of potential inhibitor structures are known to the skilled worker. In principle, an optimization of the compound identified by the abovementioned methods is also possible via molecular modeling.

A preferred embodiment of the method according to the invention, which is based on steps i) and ii), consists in selecting a test compound which reduces or blocks the enzymatic activity of a malate dehydrogenase, preferably MDH.

A particularly preferred embodiment of the method according to the invention, which is based on steps i) and ii), consists in selecting a test compound which reduces or blocks the enzymatic activity of a malate dehydrogenase, preferably MDH, the activity of the malate dehydrogenase, preferably MDH, incubated with the test compound being compared with the activity of a malate dehydrogenase, preferably MDH, not incubated with a test compound.

A preferred embodiment of the method based on steps i) and ii) consists in

i. expressing a malate dehydrogenase, preferably an MDH, in a transgenic organism according to the invention or growing an organism which naturally contains a malate dehydrogenase, preferably MDH,

ii. bringing the malate dehydrogenase, preferably the MDH, of step i) in the cell digest of the transgenic or nontransgenic organism, in partially purified or in homogeneously purified form, into contact with a test compound; and

iii. selecting a compound which reduces or blocks the activity of the malate dehydrogenase, preferably the MDH, the activity of the malate dehydrogenase, incubated with the test compound.

A preferred embodiment of the method based on steps i) and ii) consists in

i. expressing a malate dehydrogenase, preferably an MDH, in a transgenic organism according to the invention or growing an organism which naturally contains a malate dehydrogenase, preferably MDH,

ii. bringing the malate dehydrogenase, preferably the MDH, of step i) in the cell digest of the transgenic or nontransgenic organism, in partially purified or in homogeneously purified form, into contact with a test compound; and

iii. selecting a compound which reduces or blocks the activity of the malate dehydrogenase, preferably the MDH, the activity of the malate dehydrogenase, preferably the MDH, incubated with the test compound being compared with the activity of a malate dehydrogenase, preferably MDH, not incubated with a test compound.

The solution containing the malate dehydrogenase, preferably MDH, can consist of the lysate of the original organism or of the transgenic organism which has been transformed with an expression cassette according to the invention. If necessary, the malate dehydrogenase, preferably MDH, can be purified partially or fully via customary methods. A general overview over current protein purification techniques is described, for example, in Ausubel, F.M. et al., Current Protocols in Molecular Biology, Greene Publishing Assoc. and Wiley-Interscience (1994); ISBN 0-87969-309-6. In the case of recombinant preparation, the protein which has been fused with an affinity tag can be purified via affinity chromatography as is known to the skilled worker.

The malate dehydrogenase, preferably MDH, which is required for in vitro methods can thus be isolated either by means of heterologous expression from a transgenic organism according to the invention or from an organism containing an enzyme with the enzymatic activity, preferably biological, of a malate dehydrogenase, for example from an undesired plant, the term “undesired plant” being understood as meaning the species mentioned at the outset.

To identify herbicidal compounds, the malate dehydrogenase, preferably MDH, is now incubated with a test compound. After a reaction time, the enzymatic activity of the malate dehydrogenase, preferably MDH, incubated with the test compound is determined with the enzymatic activity of a malate dehydrogenase, preferably MDH, not incubated with a test compound. If the malate dehydrogenase, preferably MDH, is inhibited, a significant decrease in activity in comparison with the activity of the noninhibited polypeptide according to the invention is observed, the result being a reduction of at least 10%, advantageously at least 20%, preferably at least 30%, especially preferably by at least 50%, up to 100% reduction (blocking). Preferred is an inhibition of at least 50% at test compound concentrations of 10⁻⁴ M, preferably at 10⁻⁵ M, especially preferably of 10⁻⁶ M, based on enzyme concentration in the micromolar range.

The enzymatic activity of malate dehydrogenase, preferably MDH, can be determined for example by an activity assay in which the increase of the product, the decrease of the substrate (or starting material) or the decrease or increase of the cofactor are determined, or by a combination of at least two of the abovementioned parameters, as a function of a defined period of time.

Examples of suitable substrates are, for example, malate, (−)-tartrate(S,S), (S)-malate, 2-hydroxybutyrate, 2-hydroxy-glutarate, 2-hydroxymalonate, 2-oxobutyrate, 2-oxoglutarate, ketomalonate, L-malate, meso-tartrate(S,R), oxalacetate, and examples of suitable cofactors are NAD+, NADH, APAD, APADH. Depending on the malate dehydrogenase used, NADPH may also be employed as cosubstrate. If appropriate, derivatives of the abovementioned compounds which contain a detectable label such as, for example, a fluorescent label, a radioisotope label or a chemiluminescent label, may also be used.

The amounts of substrates to be employed in the activity tests may range between 0.5 and 100 mM, and the amounts of cofactor between 0.1 and 5 mM, based on 1-100 ∝g/ml enzyme.

The activity can be determined for example analogously to the method described by Gietl et al. (1996, BBA 1274, 48-58).

Moreover, the activity in step iii) of the abovementioned method may be determined

a) photometrically via the conversion of NADH into NAD+; and/or

b) photometrically via the conversion of NAD+to NADH, or

c) photometrically via the conversion of APAD to APADH.

A preferred embodiment of the method according to the invention which is based on steps i) and iii) consists of the following steps:

i. generation of a transgenic organism comprising a nucleic acid sequence encoding a glyoxsomal malate dehydrogenase comprising
a) a nucleic acid sequence with the nucleic acid sequence shown in SEQ ID NO:1 , or
b) a nucleic acid sequence with the nucleic acid sequence shown in SEQ ID NO:2, or
c) a nucleic acid sequence which, owing to the degeneracy of the genetic code, can be deduced from the amino acid sequence shown in SEQ ID NO:3 by backtranslating, or
d) a nucleic acid sequence which, owing to the degeneracy of the genetic code, can be deduced by backtranslating the amino acid sequence of a functional equivalent of SEQ ID NO:3, which has at least 66% identity with SEQ ID NO:3, preferably 81%,

ii. applying a test compound to the transgenic organism of i) and to a nontransgenic organism of the same genotype;

iii. determining the growth or the viability of the transgenic and the nontransgenic organisms after application of the test substance, and

iv. selecting test compounds which bring about a reduced growth or a limited viability of the nontransgenic organism in comparison with the growth of the transgenic organism.

In the abovementioned method, malate dehydrogenase is over-expressed in the transgenic organism.

In this context, the difference in growth in step iv) for the selection of a herbicidally active inhibitor amounts to at least 10%, by preference 20%, preferably 30%, especially preferably 40% and very especially preferably 50%.

The transgenic organism in this context is preferably a plant, an alga, a cyanobacterium, for example of the genus Synechocystis or a proteobacterium such as, for example, Magnetococcus sp. MC1, preferably plants which can be transformed by means of customary techniques, such as Arabidopsis thaliana, Solanum tuberosum, Nicotiana Tabacum, or cyanobacteria which can be transformed readily, such as Synechocystis, into which the sequence encoding a polypeptide according to the invention has been incorporated by transformation. These transgenic organisms thus show increased tolerance to compounds which inhibit the polypeptide according to the invention. “Knock-out” mutants in which the analogous malate dehydrogenasse, preferably MDH, gene which is naturally present in this organism has been selectively switched off may also be used.

However, the abovementioned embodiment of the method according to the invention can also be used for identifying substances with a growth-regulatory action. In this context, the transgenic organism employed is a plant. The method for identifying substances with growth-regulatory activity thus comprises the following steps:

i. generating a transgenic plant comprising a nucleic acid sequence encoding a glyoxysomal malate dehydrogenase comprising
a) a nucleic acid sequence with the nucleic acid sequence shown in SEQ ID NO:1, or
b) a nucleic acid sequence with the nucleic acid sequence shown in SEQ ID NO:2, or
c) a nucleic acid sequence which, owing to the degeneracy of the genetic code, can be deduced from the amino acid sequence shown in SEQ ID NO:3 by backtranslating, or
d) a nucleic acid which, owing to the degeneracy of the genetic code, can be deduced by backtranslating the amino acid sequence of a functional equivalent of SEQ ID NO:3, which has at least 66% identity with SEQ ID NO:3, preferably 81%,

ii. applying a test substance to the transgenic plant of i) and to a nontransgenic plant of the same variety,

iii. determining the growth or the viability of the transgenic plant and the nontransgenic plant after application of the test compound, and

iv. selecting test substances which bring about a reduced growth of the nontransgenic plant in comparison with the growth of the transgenic plant.

In the abovementioned method, malate dehydrogenase is over-expressed in the transgenic organism.

Here, step iv) involves the selection of test compounds which bring about a modified growth of the nontransgenic organism in comparison with the growth of the transgenic organism. Modified growth is understood as meaning, in this context, inhibition of the vegetative growth of the plants, which can manifest itself in particular in reduced longitudinal growth. Accordingly, the treated plants show stunted growth; moreover, their leaves are darker in color. In addition, modified growth is also understood as meaning a change in the course of maturation over time, the inhibition or promotion of lateral branched growth of the plants, shortened or extended developmental stages, increased standing ability, the growth of larger amounts of buds, flowers, leaves, fruits, seed kernels, roots and tubers, an increased sugar content in plants such as sugarbeet, sugar cane and citrus fruit, an increased protein content in plants such as cereals or soybean, or stimulation of the latex flow in rubber trees. The skilled worker is familiar with the detection of such modified growth.

It is also possible, in the method according to the invention, to employ a plurality of test compounds in a method according to the invention. If a group of test compounds affect the target, then it is either possible directly to isolate the individual test compounds or to divide the group of test compounds into a variety of subgroups, for example when it consists of a multiplicity of different components, in order to thus reduce the number of the different test compounds in the method according to the invention. The method according to the invention is then repeated with the individual test compound or the relevant subgroup of test compounds. Depending on the complexity of the sample, the above-described steps can be carried out repeatedly, preferably until the subgroup identified in accordance with the method according to the invention only comprises a small number of test compounds, or indeed just one test compound.

All of the above-described methods for identifying inhibitors with herbicidal or growth-regulatory activity are hereinbelow referred to as “methods according to the invention”.

All of the compounds which have been identified via the methods according to the invention can subsequently be tested in vivo for their herbicidal and growth-regulatory activity. One possibility of testing the compounds for herbicidal action is to use duckweed, Lemna minor, in microtiter plates. Parameters which can be measured are changes in the chlorophyll content and the photosynthesis rate. It is also possible to apply the compound directly to undesired plants, it being possible to identify the herbicidal action for example via-restricted growth.

The method according to the invention can advantageously also be carried out in high-throughput methods, known as HTS, which makes possible the simultaneous testing of a multiplicity of different compounds.

The use of supports which contain one or more of the nucleic acid molecules according to the invention, one or more of the vectors containing the nucleic acid sequence according to the invention, one or more transgenic organisms containing at least one of the nucleic acid sequences according to the invention or one or more (poly)peptides encoded via the nucleic acid sequences according to the invention lends itself to carrying out HTS in practice. The support used can be solid or liquid, but is preferably solid and especially preferably a microtiter plate. The abovementioned supports also form part of the subject matter of the present invention. In accordance with the most widely used technique, 96-well, 384-well and 1536-well microtiter plates which, as a rule, can comprise volumes of 200 μl, are used. Besides the microtiter plates, the further components of an HTS system which match the corresponding microtiter plates, such as a large number of instruments, materials, automatic pipetting devices, robots, automated plate readers and plate washers, are commercially available.

In addition to the HTS systems based on microtiter plates, what are known as “free-format assays” or assay systems where no physical barriers exist between the samples, as, for example, in Jayaickreme et al., Proc. Natl. Acad. Sci U.S.A. 19 (1994) 161418; Chelsky, “Strategies for Screening Combinatorial Libraries”, First Annual Conference of The Society for Biomolecular Screening in Philadelphia, Pa. (Nov. 710, 1995); Salmon et al., Molecular Diversity 2 (1996), 5763 and U.S. Pat. No. 5,976,813, may also be used.

The invention furthermore relates to herbicidally active compounds identified by the methods according to the invention. These compounds are hereinbelow referred to as “selected compounds”. They have a molecular weight of less than 1000 g/mol, advantageously less than 500 g/mol, preferably less than 400 g/mol, especially preferably less than 300 g/mol. Herbicidally active compounds have a Ki value of less than 1 mM, preferably less than 1 μM, especially preferably less than 0.1 μM, very especially preferably less than 0.01 μM.

The invention furthermore relates to compounds with growth-regulatory activity identified by the methods according to the invention. These compounds too are hereinbelow referred to as “selected compounds”.

Naturally, the selected compounds can also be present in the form of their agriculturally useful salts. Agriculturally useful salts which are suitable are mainly the salts of those cations, or the acid addition salts of those acids, whose cations, or anions, do not adversely affect the herbicidal action of the herbicidally active compounds identified via the methods according to the invention.

If the selected compounds contain asymmetrically substituted α-carbon atoms, they may furthermore also be present in the form of racemates, enantiomer mixtures, pure enantiomers or, if they have chiral substituents, also in the form of diastereomer mixtures.

The selected compounds can be chemically synthesized substances or substances produced by microbes and can be found, for example, in cell extracts of, for example, plants, animals or microorganisms. The reaction mixture can be a cell-free extract or comprise a cell or cell culture. Suitable methods are known to the skilled worker and are described generally for example in Alberts, Molecular Biology the cell, 3^{rd} Edition (1994), for example chapter 17. The selected compounds may also originate from comprehensive substance libraries.

Candidate test compounds can be expression libraries such as, for example, cDNA expression libraries, peptides, proteins, nucleic acids, antibodies, small organic substances, hormones, PNAs or the like (Milner, Nature Medicin 1 (1995), 879-880; Hupp, Cell. 83 (1995), 237-245; Gibbs, Cell. 79 (1994), 193-198 and references cited therein).

The selected compounds can be used for controlling undesired vegetation and/or as growth regulators. Herbicidal compositions comprising the selected compounds afford very good control of vegetation on noncrop areas. In crops such as wheat, rice, maize, soybean and cotton, they act against broad-leaved weeds and grass weeds without inflicting any significant damage on the crop plants. This effect is observed in particular at low application rates. The selected compounds can be used for controlling the harmful plants which have already been mentioned above.

Depending on the application method in question, selected compounds, or herbicidal compositions comprising them, can advantageously also be employed in a further number of crop plants for eliminating undesired plants. Examples of suitable crops are:

Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pisum sativum, Prunus avium, Prunus persica, Pyrus communis, Ribes sylvestre, Ricinus communis, Saccharum officinarum, Secale cereale, Solanum tuberosum, Sorghum bicolor (s. vulgare), Theobroma cacao, Trifolium pratense, Triticum aestivum, Triticum durum, Vicia faba, Vitis vinifera, Zea mays.

In addition, the selected compounds can also be used in crops which tolerate the action of herbicides owing to breeding, including recombinant methods. The generation of such crops is described hereinbelow.

The invention furthermore relates to a method of preparing the herbicidal or growth-regulatory composition which has already been mentioned above, which comprises formulating selected compounds with suitable auxiliaries to give crop protection products.

The selected compounds can be formulated for example in the form of directly sprayable aqueous solutions, powders, suspensions, also highly concentrated aqueous, oily or other suspensions or suspoemulsions or dispersions, emulsifiable concentrates, emulsions, oil dispersions, pastes, dusts, materials for spreading or granules, and applied by means of spraying, atomizing, dusting, spreading or pouring. The use forms depend on the intended use and the nature of the selected compounds; in any case, they should guarantee the finest possible distribution of the selected compounds. The herbicidal compositions comprise a herbicidally active amount of at least one selected compound and auxiliaries conventionally used in the formulation of herbicidal compositions.

For the preparation of emulsions, pastes or aqueous or oily formulations and dispersible concentrates (DC), the selected compounds can be dissolved or dispersed in an oil or solvent, it being possible to add further formulation auxiliaries for homogenization. However, it is also possible to prepare liquid or solid concentrates from selected compound, if appropriate solvents or oil and, optionally, further auxiliaries comprising liquid or solid concentrates, and these concentrates are suitable for dilution with water. The following can be mentioned: emulsifiable concentrates (EC, EW), suspensions (SC), soluble concentrates (SL), dispersible concentrates (DC), pastes, pills, wettable powders or granules, it being possible for the solid formulations either to be soluble or dispersible (wettable) in water. In addition, suitable powders or granules or tablets can additionally be provided with a solid coating which prevents abrasion or premature release of the active ingredient.

In principle, the term “auxiliaries” is understood as meaning the following classes of compounds: antifoams, thickeners, wetting agents, tackifiers, dispersants, emulsifiers, bactericides and/or thixotropic agents. The skilled worker is familiar with the meaning of the abovementioned agents.

SLs, EWs and ECs can be prepared by simply mixing the ingredients in question; powders can be prepared by mixing or grinding in specific types of mills (for example hammer mills). DCs, SCs and SEs are usually prepared by wet milling, it being possible to prepare an SE from an SC by addition of an organic phase which may comprise further auxiliaries or selected compounds. The preparation is known. Powders, materials for spreading and dusts can advantageously be prepared by mixing or cogrinding the active substances together with a solid carrier. Granules, for example coated granules, impregnated granules and homogeneous granules, can be prepared by binding the selected compounds to solid carriers. The skilled worker is familiar with further details regarding their preparation, which are mentioned for example in the following publications: U.S. Pat. No. 3,060,084, EP-A 707445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. Nos. 4,172,714, 4,144,050, 3,920,442, 5,180,587, 5,232,701, 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Federal Republic of Germany), 2001.

The skilled worker is familiar with a multiplicity of inert liquid and/or solid carriers which are suitable for the formulations according to the invention, such as, for example, liquid additives such as mineral oil fractions of medium to high boiling point such as kerosene or diesel oil, furthermore coal tar oils and oils of vegetable or animal origin, aliphatic, cyclic and aromatic hydrocarbons, for example paraffin, tetrahydrophthalene, alkylated naphthalenes or their derivatives, alkylated benzenes or their derivatives, alcohols such as methanol, ethanol, propanol, butanol and cyclohexanol, ketones such as cyclohexanone, or strongly polar solvents, for example amines such as N-methylpyrrolidone or water.

Examples of solid carriers are mineral earths such as silicas, silica gels, silicates, talc, kaolin, limestone, lime, chalk, bole, loess, clay, dolomite, diatomaceous earth, calcium sulfate, magnesium sulfate, magnesium oxide, ground synthetic materials, fertilizers such as ammonium sulfate, ammonium phosphate, ammonium nitrate, ureas and products of vegetable origin such as cereal meal, tree bark meal, wood meal and nutshell meal, cellulose powders or other solid carriers.

The skilled worker is familiar with the multiplicity of surface-active substances (surfactants) which are suitable for the formulations according to the invention such as, for example, alkali metal salts, alkaline earth metal salts or ammonium salts of aromatic sulfonic acids for example lignosulfonic acid, phenolsulfonic acid, naphthalenesulfonic acid, and dibutylnaphthalenesulfonic acid, and of fatty acids, of alkyl- and alkylarylsulfonates, of alkyl sulfates, lauryl ether sulfates and fatty alcohol sulfates, and salts of sulfated hexa-, hepta- and octadecanols and of fatty alcohol glycol ethers, condensates of sulfonated naphthalene and its derivatives with formaldehyde, condensates of naphthalene or of the naphthalenesulfonic acids with phenol and formaldehyde, polyoxyethylene octylphenol ether, ethoxylated isooctyl-, octyl- or nonylphenol, alkylphenyl polyglycol ethers, tributylphenyl polyglycol ether, alkylaryl polyether alcohols, isotridecyl alcohol, fatty alcohol/ethylene oxide condensates, ethoxylated caster oil, polyoxyethylene alkyl ethers or polyoxypropylene alkyl ethers, lauryl alcohol polyglycol ether acetate, sorbitol esters, lignosulfite waste liquors or methylcellulose.

The herbicidal compositions, or the selected compounds, can be applied pre- or post-emergence. If the selected compounds are less well tolerated by certain crop plants, application techniques may be used in which the selected compounds are sprayed, with the aid of the spraying apparatus, in such a way that they come into as little contact, if any, with the leaves of the sensitive crop plants while the selected compounds reach the leaves of undesired plants which grow underneath, or the bare soil surface (post-directed, lay-by).

Depending on the intended purpose of the control measures, the season, the target plants and the growth stage, the application rates of selected compounds amount to 0.001 to 3.0, preferably 0.0 to 1.0 kg/ha.

Providing the herbicidal target furthermore makes possible a method for identifying a malate dehydrogenase, preferably MDH, which is not, or only to some extent, inhibited by a herbicide which has malate dehydrogenase, preferably MDH, as its site of action, for example the herbicidally active compounds which have been selected. Hereafter, a protein which differs in such a way from malate dehydrogenase, preferably MDH, is referred to as MDH variant, which is encoded by a nucleic acid sequence which

i) encodes a polypeptide with the biological activity of a malate dehydrogenase which is not inhibited by herbicidally active substances identified by the above-mentioned methods which inhibit malate dehydrogenase, preferably MDH;

ii) are encoded by a functional equivalent of the nucleic acid sequence SEQ ID NO:3 with at least 63% identity with SEQ ID NO:3.

In a preferred embodiment, the abovementioned method for generating nucleic acid sequences encoding MDH variants of nucleic acid sequences comprise the following steps:

a) expression of the proteins encoded by the abovementioned nucleic acids in a heterologous system or in a cell-free system;

b) randomized or directed mutagenesis of the protein by modification of the nucleic acid;

c) measuring the interaction of the modified gene product with the herbicide;

d) identification of derivatives of the protein which show less interaction;

e) assaying the biological activity of the protein after application of the herbicide;

f) selection of the nucleic acid sequences which have modified biological activity with regard to the herbicide.

The functional equivalent of SEQ ID NO:3 in accordance with ii) has at least 63%, 64%, 65%, 66%, by preference at least 67%, 68%, 69%, 70%, 71%, 72%, 73%, 74%, by preference at least 75%, 76%, 77%, 78%, 79%, 80%, preferably at least 81%, 82%, 83%, 84%, 85%, 86%, 87%, 88%, 89%, 89%, 90%, 91%, 92%, 93%, especially preferably at least 94%, 95%, 96%, 97%, 98%, 99% homology with SEQ ID NO:3.

The sequences selected by the above-described method are advantageously introduced into an organism. This is why the invention furthermore relates to an organism prepared by this method. The organism is preferably a. plant, especially preferably one of the above-defined crop plants.

Thereafter, intact plants are regenerated, and the resistance to the selected compound is verified in intact plants.

Modified proteins and/or nucleic acids which are capable of mediating, in plants, resistance to the selected compounds can also be prepared from the above-mentioned nucleic acid sequences via what is known as “site-directed mutagenesis”; by means of this mutagenesis, for example the stability and/or activity of the target protein or the characteristics such as binding of the abovementioned inhibitors according to the invention can be improved or modified in a highly specific manner.

For example, Zhu et al. (Nature Biotech., Vol. 18, May 2000: 555-558) have described a site-directed mutagenesis method in plants which can be used advantageously.

Moreover, modifications can be achieved via the PCR method described by Spee et al. (Nucleic Acids Research, Vol. 21, No. 3, 1993: 777-78) using dITP for the random mutagenesis, or by the further-improved method of Rellos et al. (Protein Expr. Purif., 5, 1994: 270-277).

A further possibility of producing these modified proteins and/or nucleic acids is an in-vitro recombination technique for molecular evolution which has been described by Stemmer et al. (Proc. Natl. Acad. Sci. USA, Vol. 91, 1994: 10747-10751), or the combination of the PCR and recombination method described by Moore et al. (Nature Biotechnology Vol. 14, 1996: 458-467).

A further way for the mutagenesis of proteins is described by Greener et al. in Methods in Molecular Biology (Vol. 57, 1996: 375-385). A method for modifying proteins using the microorganism E. coli XL-1 Red is described in EP-A-0 909 821. During its replication, this microorganism generates mutations in the nucleic acids which have been introduced and thus leads to a modification of the genetic information. Advantageous nucleic acids and the proteins encoded by them can be identified readily by isolating the modified nucleic acids or the modified proteins and carrying out resistance tests. The former can then be introduced into plants, where they are capable of manifesting the resistance and thus lead to resistance to the herbicides.

Further methods of mutagenesis and selection are, for example, methods such as the in vivo mutagenesis of seeds or pollen and the selection of resistant alleles in the presence of the inhibitors according to the invention, followed by genetic and molecular identification of the modified, resistant allele and also the mutagenesis and selection of resistances in cell culture by propagating the culture in the presence of successively increasing concentrations of the inhibitors according to the invention. In this context, the increase of the spontaneous mutation rate by chemical/physical mutagenic treatment may be exploited. As described above, modified genes can also be isolated using microorganisms which have an endogenous or recombinant activity of the proteins encoded by the nucleic acids used in the method according to the invention, and which are sensitive to the inhibitors identified in accordance with the invention. Growing the microorganisms on media with increasing concentrations of inhibitors according to the invention permits the selection and evolution of resistant variants of the targets according to the invention. The mutation frequency, in turn, can be increased by mutagenic treatments.

Methods for the specific modification of nucleic acids are furthermore available (Zhu et al. Proc. Natl. Acad. Sci. USA, Vol. 96, 8768-8773 and Beethem et al., Proc. Natl. Acad. Sci. USA, Vol. 96, 8774-8778). These methods make possible the replacement, in the proteins, of those amino acids which are important for the binding of inhibitors by functionally analogous amino acids which, however, prevent the inhibitor from binding.

The invention furthermore therefore relates to a method for establishing nucleic acid sequences which encode gene products which have a modified biological activity, the biological activity having been modified in such a way that an increased activity is present. An increased activity is understood as meaning an activity which is increased by at least 10%, preferably by at least 30%, especially preferably by at least 50%, very especially preferably by at least 100% in comparison with the starting organism, or the starting gene product. Moreover, the biological activity can have been modified in such a way that the substances and/or compositions according to the invention no longer bind, or no longer properly bind, to the nucleic acid sequences and/or the gene products encoded by them. For the purposes of the invention, no longer, or no longer properly, is to be understood as meaning that the substances bind by at least 30% less, preferably at least 50% less, especially preferably by at least 70% less, very especially preferably by at least 80% less, or indeed no longer, to the modified nucleic acids and/or gene products in comparison with the starting gene product or the starting nucleic acids.

Yet a further aspect of the invention therefore relates to a transgenic plant which has been transformed with a nucleic acid sequence which encodes a gene product with a modified biological activity, or with a nucleic acid sequence encoding an MDH variant. Transformation methods are known to the skilled worker and detailed further above by way of example.

Genetically modified transgenic plants which are resistant to substances found in accordance with the methods according to the invention and/or to compositions comprising these substances can also be generated by transformation, followed by overexpression of a nucleic acid sequence according to the invention. The invention therefore furthermore relates to a method for the generation of transgenic plants which are resistant to substances found by a method according to the invention, which comprises overexpressing, in these plants, nucleic acids encoding a malate dehydrogenase, preferably MDH. A similar method is described by way of example in Lermantova et al., Plant Physiol., 122, 2000: 75- 83.

The above-described methods according to the invention for generating resistant plants make possible the development of novel herbicides which have as complete as possible an action which is independent of the plant species (what are known as nonselective herbicides), in combination with the development of useful plants which are resistant to the nonselective herbicide. Useful plants which are resistant to nonselective herbicides have already been described on several occasions. In this context, one can distinguish between several principles for achieving a resistance:

a) Generation of resistance in a plant via mutation methods or recombinant methods by markedly overproducing the protein which acts as target for the herbicide and by the fact that, owing to the large excess of the protein which acts as target for the herbicide, the function exerted by this protein in the cell is even retained after application of the herbicide.

b) Modification of the plant such that a modified version of the protein which acts as target of the herbicide is introduced and that the function of the newly introduced modified protein is not adversely affected by the herbicide.

c) Modification of the plant such that a novel protein/a novel RNA is introduced wherein the chemical structure of the protein or of the nucleic acid, such as of the RNA or the DNA, which structure is responsible for the herbicidal action of the low-molecular-weight substance, is modified so that, owing to the modified structure, a herbicidal action can no longer be developed, that is to say that the interaction of the herbicide with the target can no longer take place.

d) The function of the target is replaced by a novel gene introduced into the plant, creating what is known as an alternative pathway.

e) The function of the target is taken over by another gene which is present in the plant, or by its gene product.

The skilled worker is familiar with alternative methods for identifying the homologous nucleic acids, for example in other plants, with similar sequences, such as, for example, using transposons. The present invention therefore also relates to the use of alternative insertion mutagenesis methods for the insertion, into other plants, of foreign nucleic acids into the nucleic acid sequence SEQ ID NO:3 into sequences derived from these sequences owing to the genetic code and/or derivatives of such sequences.

The transgenic plants are generated by customary transformation methods which have been described above, using one of the likewise above-described embodiments of the expression cassettes according to the invention.

The expression efficacy of the :recombinantly expressed MDH variant can be determined for example in vitro by shoot meristem propagation or by a germination test.

Moreover, an expression of the malate dehydrogenase, preferably MDH, gene, which has been modified in terms of nature and level, and its effect on the resistance to malate dehydrogenase, preferably MDH, inhibitors can be tested on plants in greenhouse experiments.

The invention is illustrated in greater detail by the examples which follow, which are not to be considered as limiting.

### EXAMPLE 1

### Generation of a cDNA Library in the Plant Transformation Vector

To generate a cDNA library (hereinbelow termed “binary cDNA library”) in a vector which can be used directly for transforming plants, mRNA was isolated from a variety of plant tissues and transcribed into double-stranded cDNA using the cDNA Synthese Kit (Amersham Pharmacia Biotech, Freiburg). The cDNA first-strand synthesis was carried out using T12-18 oligonucleotides following the manufacturer's instructions. After size fractionation and the ligation of EcoRI-NotI adapters following the manufacturer's instructions and filling up the overhangs with Pfu DNA polymerase (Stratagene), the cDNA population was normalized. To this end, the method of Kohci et al., 1995, Plant Journal 8, 771-776 was followed, the cDNA being amplified by PCR with the oligonucleotide N1 under the conditions given in Table 1.

TABLE 1

| Temperature [° C.] | Time [sec] | Number of cycles |
|---|---|---|
| 94 | 300 | 1 |
| 94 | 8 | 10 |
| 52 | 60 | |
| 72 | 180 | |
| 94 | 8 | 10 |
| 50 | 60 | |
| 72 | 180 | |
| 94 | 8 | 10 |
| 48 | 60 | |
| 72 | 180 | |
| 72 | 420 | 1 |

The resulting PCR product was bound to the column matrix of the PCR purification kit (Qiagen, Hilden) and eluted with 300 mM NaP buffer, pH 7.0, 0.5 mM EDTA, 0.04% SDS. The DNA was denatured for 5 minutes in a boiling water bath and subsequently renatured for 24 hours at 60° C. 50 μl of the DNA were applied to a hydroxylapatite column and the column was washed 3 times with 1 ml of 10 mM NaP buffer, pH 6.8. The bound single-stranded DNA was eluted with 130 mM NaP buffer, pH 6.8, precipitated with ethanol and dissolved in 40 μl of water. 20 μl thereof were used for a further PCR amplification as described above. After further ssDNA concentration, a third PCR amplification was carried out as described above.

The plant transformation vector for taking up the cDNA population which had been generated as described above was generated via restriction enzyme cleavage of the vector pUC18 with SbfI and BamHI, purification of the vector fragment followed by filling up the overhangs with Pfu DNA polymerase and religation with T4 DNA ligase (Stratagene). The resulting construct is hereinbelow termed pUC18SbfI-.

The vector pBinAR was first cleaved with NotI, the ends were filled up and the vector was religated, cleaved with SbfI, the ends were filled up and the vector was religated and subsequently cleaved with EcoRI and HindIII. The resulting fragment was ligated into a derivative of the binary plant transformation vector pPZP (Hajdukiewicz, P, Svab, Z, Maliga, P., (1994) Plant Mol Biol 25:989-994) which makes possible the transformation of plants by means of agrobacterium and mediates kanamycin resistance in transgenic plants. The construct generated thus is hereinbelow termed pSun12/35S. pUC18SbfI- was used as template in a polymerase chain reaction (PCR) with the oligonucleotides V1 and V2 (see Table 2) and Pfu DNA polymerase. The resulting fragment was ligated into the SmaI-cut pSun12/35S, giving rise to pSunblues2. Following cleavage with NotI, dephosphorylation with shrimp alkaline phosphatase (Roche Diagnostics, Mannheim) and purification of the vector fragment, pSunblues2 was ligated with the normalized, likewise NotI-cut cDNA population. Following transformation into E. coli Xl-1blue (Stratagene), the resulting clones were deposited into microtiter plates. The binary cDNA library contains cDNAs in “sense” and in “antisense” orientation under the control of the cauliflower mosaic virus 35S promoter, and, after transformation into tobacco plants, these cDNAs can, accordingly, lead to “cosuppression” and “antisense” effects.

TABLE 2
Oligonucleotides used

| | |
|---|---|
| Oligo- | |
| nucleotide | Nucleic acid sequence |

| | | |
|---|---|---|
| N1 | 5′-AGAATTCGCGGCCGCT-3′ | (SEQ ID NO:4) |
| V1 | 5′-CTCATGCGGCCGCGCGCAACGCA | (SEQ ID NO:5) |
| (PWL93not) | ATTAATGTG-3′ | |
| V2 (pWL92) | 5′-TCATGCGGCCGCGAGATCCAGTT | (SEQ ID NO:6) |
| | CGATGTAAC-3′ | |
| G1 (35S) | 5′-GTGGATTGATGTGATAT | (SEQ ID NO:7) |
| | CTCC-3′ | |
| G2 (OCS) | 5′-GTAAGGATCTGAGCTAC | (SEQ ID NO:8) |
| | ACAT-3′ | |

### EXAMPLE 2

### Transformation and Analysis of Tobacco Plants

Selected clones of the binary cDNA library were transformed into Agrobacterium tumefaciens C58C1:pGV2260 and (Deblaere et al., Nucl. Acids. Res. 13(1984), 4777-4788) and incubated with Streptomycin/Spectinomycin selection. The material used for the transformation of tobacco plants (Nicotiana tabacum cv. Samsun NN) with the clone nt002035041r was an overnight culture of a positively transformed agrobacterial colony diluted with YEB medium to OD600=0.8-1.6. Leaf discs of sterile plants (approx. 1 cm² each) were incubated for 5-10 minutes with the agrobacterial dilution in a Petri dish. This was followed by incubation in the dark for 2 days at 25° C. on Murashige-Skoog medium (Physiol. Plant. 15(1962), 473) supplemented with 2% sucrose (2MS medium) and 0.8% Bacto agar. The cultivation was continued after 2 days with 16 hours of light/8 hours of darkness and continued in a weekly rhythm on MS medium supplemented with 500 mg/l Claforan (cefotaxime sodium), 50 mg/l kanamycin, 1 mg/l benzylaminopurine (BAP), 0.2 mg/l naphthylacetic acid and 1.6 g/l glucose. Growing shoots were transferred onto MS medium supplemented with 2% sucrose, 250 mg/l Claforan and 0.8% Bacto agar. Regenerated shoots were transferred onto 2MS medium supplemented with kanamycin and Claforan. Transgenic plants of line E_{—}0000005869 were generated in this manner.

The integration of the clone cDNA into the genome of the transgenic lines was detected via PCR with the oligonucleotides G1 and G2 (see Table 2) and genomic DNA prepared from the transgenic lines in question. To this end, TAKARA Taq DNA polymerase was preferably employed, following the manufacturer's instructions (MoBiTec, Göttingen). The cDNA clone of the binary cDNA library, which clone had been used in each case for the transformation, acted as template for a PCR reaction as the positive control. PCR products with an identical size or, if appropriate, identical cleavage patterns which were obtained after cleavage with a variety of restriction enzymes acted as proof that the corresponding cDNA had been integrated. In this manner, the insert of clone nt002035041r was detected in the transgenic plants with the abovementioned phenotypes.

After the shoots had been transferred into soil, the plants were observed for 2-20 weeks in the greenhouse for the manifestation of phenotypes. It emerged that transgenic plants of line E_{—}0000005869, in which the nt002035041r insert had been detected, were similar in phenotype. These plants showed pronounced growth retardation after 2 weeks and also chlorotic leaves and, in some cases, necroses.

### EXAMPLE 3

### Sequence Analysis of the Clones

The Smart-RACE kit (Clontech) was employed following the manufacturer's instructions to isolate a full-length cDNA sequence of nt002035041r (SEQ ID NO:2). The 1513 bp SEQ ID NO:2 encodes a protein of 320 amino acids on an open reading frame (nt 148-1221) which is 960 nt in length.

Thus, it has been shown for the first time and in a surprising manner that the natural expression of sequences encoding glyoxysomal MDH is essential for plants, and that reduced expression leads to damage as can be seen on the phenotypes mentioned in Example 2. It can thus be concluded that malate dehydrogenase, preferably MDH, is suitable as target for herbicides.

The highest degree of homology is with glyoxysomal MDHs from watermelon (P19446, Swissprot) with 77.5% identity at the nucleotide level (90.7% similarity, or 85.4% identity, at the protein level), cucumber, Arabidopsis, soybean and rice. The N-terminal 38 amino acids are presumably a glyoxysomal transit sequence.

### EXAMPLE 4

### Expression in E. coli

To generate active Nt-MDH protein with plant MDH activity, the coding regions Nt-MDH and MDH from Arabidopsis (Genbank: At2g22780) was overexpressed in E. coli bacteria. To this end, Nt-MDH fragments were amplified by PCR using specific oligonucleotides and Pfu-Ultra Polymerase (Stratagene) and ligated into the expression vector pCR T7/CT TOPO (Invitrogen) (constructs Nt1 and At1, Table 7). In this manner, encoded fusion proteins with C-terminal hexahistidine tags were generated. The use of the specified oligonucleotides in the cloning step gave N-terminally truncated versions of MDH in order to exclude the glyoxysomal transit sequence, which might counteract functional expression. The starting point was the transit sequence of the watermelon glyoxysomal MDH (Gietl. et al. 1996, BBA 1274, 48-58). The PCR was carried out in 36 cycles following standard conditions (for example as described by Sambrook, J. et al. (1989) “Molecular cloning: A laboratory manual”, Cold Spring Harbor Laboratory Press), the annealing temperatures being between 45 and 55° C. and the polymerization time being in each case 60 seconds per 1000bp. The templates and the primers used for the template in question are shown in Table 7 together with the annealing temperatures.

TABLE 7

| | | |
|---|---|---|
| | Annealing | |
| | temperature | |
| Construct | [°C.] | Primer (Nucleic acid sequence) |

| | | | |
|---|---|---|---|
| Nt-gMDH-E1⁽¹⁾ | 53 | 5′-ATGAGGGCGAAAGG | (SEQ ID NO:9) |
| | | GGG-3′ | |
| | | 5′-TTTCTTTACAAAGTTGAC | (SEQ ID NO:10) |
| | | ACCCTTC-3′ | |
| At-gMDH-E1⁽²⁾ | 56 | 5′-ATGCGGGCAAAAGG | (SEQ ID NO:11) |
| | | TGG-3′ | |
| | | 5′-TTTCTTCGCAAAGGTAAC | (SEQ ID NO:12 |
| | | ACC-3′ | |

⁽¹⁾Template: Nicotiana tabacuum cDNA library
⁽²⁾Template: Arabidopsis thaliana cDNA library

The PCR products were ligated into the vector pCR T7/CT TOP and transformed into E. coli. Expression was performed in E. coli BL21(DE3) strains such as BL21(DE3)pLysS or BL21(DE3)pLysE (Invitrogen), BL21-CodonPlus(DE3) or BL21-CodonPlus(DE3)RIL (Stratagene) following induction with IPTG. Standard protocols (Invitrogen) were followed.

The expression products of Nt-gMDH-E1 and At-gMDH-E1 were purified by affinity chromatography on Ni-agarose. The manufacturer's instructions were followed (Qiagen).

### EXAMPLE 5

### In Vitro Assay Systems

The enzyme activity of gMDH can be measured in preparations of plant microbodies by the method of Huang et al. (1974, Plant Physiol 54, 364-368). However, an advantageous way of proceeding is to employ the proteins expressed in E. coli and purified as described above in order to minimize background activities of cytosolic and mitochondrial MDHs.

The enzymatic activity of the gMDH of Example 3 was measured photometrically via the conversion of NAD+ to NADH at 340 nm by the method of Gietl et al. (1996, BBA 1274, 48-58). This assay can be carried out on the microtiter plate scale.

1-26. (canceled)

27. A glyoxysomal malate dehydrogenase which is encoded by a nucleic acid sequence comprising:
a) a nucleic acid sequence with the nucleic acid sequence shown in SEQ ID NO:1, or
b) a nucleic acid sequence with the nucleic acid sequence shown in SEQ ID NO:2, or
c) a nucleic acid sequence which, owing to the degeneracy of the genetic code, can be deduced from the amino acid sequence shown in SEQ ID NO:3 by backtranslating, or
d) a nucleic acid sequence which, owing to the degeneracy of the genetic code, can be deduced by backtranslating the amino acid sequence of a functional equivalent of SEQ ID NO:3, which has at least 66% identity with SEQ ID NO:3.

28. A plant nucleic acid sequence encoding a glyoxysomal malate dehydrogenase comprising:
a) a nucleic acid sequence with the nucleic acid sequence shown in SEQ ID NO:1, or
b) a nucleic acid sequence with the nucleic acid sequence shown in SEQ ID NO:2, or
c) a nucleic acid sequence which, owing to the degeneracy of the genetic code, can be deduced from the amino acid sequence shown in SEQ ID NO:3 by backtranslating, or
d) a nucleic acid sequence which, owing to the degeneracy of the genetic code, can be deduced by backtranslating the amino acid sequence of a functional equivalent of SEQ ID NO:3, which has at least 79% identity with SEQ ID NO:3, or
e) a nucleic acid sequence which, owing to the degeneracy of the genetic code, can be deduced by backtranslating the amino acid sequence of a functional equivalent of SEQ ID NO:3, which has at least 87% identity with SEQ ID NO:3.

29. A polypeptide with the biological activity of a glyoxysomal malate dehydrogenase as target for herbicides, encoded by a nucleic acid molecule according to claim 28.

30. A method for detecting functional analogs of SEQ ID NO:2 comprising
a) generating a probe followed by subsequently screening a genomic library or cDNA library of the species in question, or
b) conducting a computer search for analogous sequences in electronic databases.

31. An expression cassette comprising genetic control sequences in operable linkage with a nucleic acid sequence according to claim 28.

32. A vector comprising an expression cassette according to claim 31.

33. A transgenic organism comprising at least one nucleic acid sequence encoding a polypeptide with the biological activity of a glyoxysomal malate dehydrogenase according to claim 28, an expression cassette according to claim 31 or a vector according to claim 32, selected from among bacteria, yeasts, fungi, animal cells or plant cells.

34. A method for identifying substances with herbicidal activity, comprising the following steps:
i. bringing malate dehydrogenase into contact with one or more test compounds under conditions which permit the test compound(s) to bind to the nucleic acid molecule or to the glyoxysomal malate dehydrogenase, and
ii. detecting whether the test compound binds to the malate dehydrogenase of i), or
iii. detecting whether the test compound reduces or blocks the enzymatic or biological activity of the malate dehydrogenase of i), or
iv. detecting whether the test compound reduces or blocks the transcription, translation or expression of the malate dehydrogenase of i),
the malate dehydrogenase being encoded by a nucleic acid sequence which comprises
a) a nucleic acid sequence according to claim 28, or
b) a nucleic acid sequence which, owing to the degeneracy of the genetic code, can be deduced by backtranslating the amino acid sequence of a functional equivalent of SEQ ID NO:3, which has at least 63% identity with SEQ ID NO:3.

35. A method according to claim 34, wherein the malate dehydrogenase is a glyoxysomal malate dehydrogenase and is encoded by a nucleic acid sequence which comprises
a) a nucleic acid sequence according to claim 28, or
b) a nucleic acid sequence which, owing to the degeneracy of the genetic code, can be deduced by backtranslating the amino acid sequence of a functional equivalent of SEQ ID NO:3, which has at least 66% identity with SEQ ID NO:3.

36. A method according to claim 34, wherein a test compound is selected which reduces or blocks the enzymatic or biological activity of the glyoxysomal malate dehydrogenase.

37. A method according to claim 34, wherein
i. either malate dehydrogenase is expressed in a transgenic organism or an organism which naturally contains malate dehydrogenase is grown,
ii. the malate dehydrogenase of step i) is brought into contact with a test compound in the cell digest of the transgenic or nontransgenic organism, in partially purified form or in homogeneously purified form, and
iii. a test compound which reduces or blocks the enzymatic activity of the malate dehydrogenase of step a) is selected.

38. A method according to claim 34, which comprises the following steps:
i. generating a transgenic organism according to claim 33 or a transgenic organism comprising a nucleic acid sequence encoding a malate dehydrogenase comprising a nucleic acid sequence which, owing to the degeneracy of the genetic code, can be deduced by backtranslating the amino acid sequence of a functional equivalent of SEQ ID NO:3, which has at least 63% identity with SEQ ID NO:3,
malate dehydrogenase being overexpressed in the transgenic organism,
ii. applying a test substance to the transgenic plant of i) and to a nontransgenic plant of the same genotype,
iii. determining the growth or the viability of the transgenic plant and the nontransgenic plant after application of the test compound, and
iv. selecting test substances which bring about a reduced growth of the nontransgenic plant in comparison with the growth of the transgenic plant.

39. A method according to claim 38, which is carried out in a plant organism, a cyanobacterium or proteobacterium.

40. A method for identifying substances with growth-regulatory activity, which comprises the following steps:
i. generating a transgenic plant comprising a nucleic acid sequence encoding a polypeptide with the biological activity of a glyoxysomal malate dehydrogenase comprising
a) a nucleic acid sequence according to claim 28, or
b) a nucleic acid sequence which, owing to the degeneracy of the genetic code, can be deduced by backtranslating the amino acid sequence of a functional equivalent of SEQ ID NO:3 which has at least 63% identity with SEQ ID NO:3,
malate dehydrogenase being overexpressed in the transgenic organism,
ii. applying a test substance to the transgenic plant of i) and to a nontransgenic plant of the same variety,
iii. determining the growth or the viability of the transgenic plant and the nontransgenic plant after application of the test compound, and
iv. selecting test substances which bring about a reduced growth of the nontransgenic plant in comparison with the growth of the transgenic plant.

41. A method according to claim 34, wherein the substances are identified in high-throughput screening method.

42. A support comprising one or more of the nucleic acid molecules according to claim 28, one or more expression cassettes according to claim 31, one or more vectors according to claim 32, one or more organisms according to claim 33 or one or more (poly)peptides according to claim 29.

43. A method according to claim 34, wherein the substances are identified in High-Throughput Screening using a support according to claim 42.

44. A compound with herbicidal activity, identified by one of the method of claim 34.

45. A compound with growth-regulatory activity, identified by the method according to claim 40.

46. A method for the preparation of an agrochemical composition, which comprises
a) identifying a compound with herbicidal activity by the method according to claim 8, and
b) formulating this compound together with suitable auxiliaries to give crop protection products with herbicidal or growth-regulatory activity.

47. A method for controlling undesired vegetation and/or for regulating the growth of plants, which comprises allowing at least one malate dehydrogenase inhibitor according to claim 44 to act on plants, their environment and/or on seeds.

48. A method for generating nucleic acid sequences which encode malate dehydrogenase which is not inhibited by substances according to claim 44, where the nucleic acid sequence comprises a nucleic acid sequence which, owing to the degeneracy of the genetic code, can be derived by backtranslating the amino acid sequence of a functional equivalent of SEQ ID NO:3 which has at least 63% identity with SEQ ID NO:3;
which comprises the following process steps:
a) expressing the protein encoded by the nucleic acid sequence according to i) in a heterologous system or in a cell-free system;
b) performing randomized or directed mutagenesis of the protein by modification of the nucleic acid;
c) measuring the interaction of the modified gene product with the herbicide;
d) identifying derivatives of the protein which show less interaction;
e) assaying the biological activity of the protein after application of the herbicide;
f) selecting the nucleic acid sequences which have modified biological activity with regard to the herbicide.

49. The method according to claim 48, wherein the sequences selected in accordance with step f) are introduced into an organism.

50. A method for the generation of transgenic plants which are resistant to substances according to claim 44, wherein a nucleic acid sequence encoding a malate dehydrogenase which comprises
a) a nucleic acid sequence with the nucleic acid sequence shown in SEQ ID NO:1, or
b) a nucleic acid sequence with the nucleic acid sequence shown in SEQ ID NO:2, or
c) a nucleic acid sequence which, owing to the degeneracy of the genetic code, can be deduced from the amino acid sequence shown in SEQ ID NO:3 by backtranslating, or
d) a nucleic acid sequence which, owing to the degeneracy of the genetic code, can be deduced by backtranslating the amino acid sequence of a functional equivalent of SEQ ID NO:3, which has at least 79% identity with SEQ ID NO:3, or
e) a nucleic acid sequence which, owing to the degeneracy of the genetic code, can be deduced by backtranslating the amino acid sequence of a functional equivalent of SEQ ID NO:3, which has at least 87% identity with SEQ ID NO:3, or
f) a functional equivalent of the nucleic acid sequence of SEQ ID NO:3 which has at least 63% identity with SEQ ID NO:3;
is overexpressed in these plants.

51. A transgenic plant generated by a method according to claim 50.
